(19) United States
(12) Patent Application Publication
(10) Pub. No.: US 2017/0014639 A1
(43) Pub. Date: Jan. 19, 2017

(54) CABLE WITH MICROWAVE EMITTER

(71) Applicant: Symple Surgical, Inc., Flagstaff, AZ (US)

(72) Inventors: Justin Randy PRESTON, San Francisco, CA (US); Sohail DESAI, Sacramento, CA (US); Seth CROZIER, Dublin, CA (US)

(73) Assignee: Symple Surgical, Inc., Flagstaff, AZ (US)

(21) Appl. No.: 14/798,358

(22) Filed: Jul. 13, 2015

(51) Int. Cl. A61N 5/02

(52) U.S. Cl. CPC A61N 5/025

(57) ABSTRACT

A microware emitter cable system is disclosed. The system can have a coaxial cable that can have an outer conductor, dielectric insulator radially inside the outer conductor, and an inner conductor radially inside of the dielectric. The system can have multiple passageways radially inside of the outer conductor. The passageways can extend to the distal terminal end of the cable.

### BACKGROUND

1. Field of the Invention

The present disclosure relates to the field of microwave cables and emitters for use in biological lumen. More particularly, this disclosure relates to a system of microwave emitters and/or coaxial cables as part of catheters.

2. Description of Related Art

Some microwave emitters, such as antennas, are at the distal end of coaxial cables in energy delivery systems and used to heat biological tissue. FIG. 1 illustrates a layered view of a typical microwave coaxial cable. The cable has a central inner conductor surrounded by a dielectric insulator, which in turn is surrounded by an outer conductor. An insulating cable jacket then surrounds the entire cable assembly.

Some of these emitters are deployed through body lumen to position the emitters adjacent to tissue that is the target of the microwave energy. In some devices, the antennae are surrounded by an inflatable balloon. The balloon is inflated and the antenna is excited to deliver microwave energy to target tissue.

Temperature control is an issue with many of these devices, particularly the ability to fine tune the temperature of the antenna and the target tissue. Position control of the emitter within the lumen is also a concern. For example, the emitter may be intended, to be positioned centrally in the lumen to spread the energy delivery evenly around the lumen or offset to one side to deliver more energy to a particular side of the lumen. Furthermore, the emitter may be angulated either passively or non-passively to deliver energy to targeted tissue. Accordingly, fluid delivery is generally desired.

Delivery of the device to the target site is usually accomplished over a guidewire. However, the central lumen of the cable is typically used for the guidewire. Therefore the inner lumen is configured to receive and slide against the guidewire, having a distal port beyond the balloon for the guidewire to exit the lumen, rather than being configured for fluid delivery. Delivering fluid to the balloon is still known, but is accomplished through a port at the proximal end of the balloon, impairing the ability to rapidly circulate fluid through the entire balloon and maintain fine control of the temperature of the balloon, cable, emitter, and/or tissue.

Accordingly, an apparatus with the ability to deliver fluid flow to a balloon surrounding the antennae and also use a generally centrally-located guidewire is desired.

### BRIEF SUMMARY OF THE INVENTION

A system, apparatus, or device for delivering microwave energy to a target biological tissue is disclosed. The system can have a coaxial cable. The cable can have a microwave emitter, an inner conductor, and an outer conductor radially outside of and electrically insulated from the inner conductor. The coaxial cable can have a first passageway extending through the microwave emitter radially inside of a radially inner surface of the outer conductor. The coaxial cable can have at least one second passageway extending through the microwave emitter radially inside of the radially inner surface of the outer conductor.

The inner conductor can have an inner lumen. The first passageway can be in the inner lumen. The second passageway can be in the inner lumen.

The system can have a catheter. At least a length of the cable can be radially inside of the catheter. The system can have a balloon at a distal end of the catheter. The system can have a balloon longitudinally coincidental and radially outside of the microwave emitter.

The system can have a third passageway radially outside of the cable. The third passageway can be in fluid communication with the balloon.

The system can have a liner between the first passageway and the second passageway. The liner can surround the first passageway.

The system can have a guidewire in the first passageway. The guidewire and first passageway can be configured so the guidewire can longitudinally translate or slide within the first passageway (e.g., being slidably configured). The first passageway can be radially centered with respect to the cross-section of the coaxial cable. The first passageway can be radially off-center with respect to the cross-section of the coaxial cable. The system can have a fluid flowing in the second passageway. The system can have a porous material, such as sponge, in the second passageway. The second passageway can be capable of allowing fluid passage. The fluid can be a liquid and/or gas.

A further system for delivering microwave energy to a target biological tissue is disclosed. The system can have a coaxial cable having a microwave emitter. The cable can have a first passageway extending through the emitter. The first passageway can have a distal port distal to the emitter. The cable can have an actively or passively closable configuration of the first passageway distal to the emitter.

The cable can have a second passageway and the catheter can have a third passageway defined between the catheter and the coaxial cable. The third passageway can be in fluid communication with the balloon.

A further system for delivering microwave energy to a target biological tissue is disclosed. The system can have a coaxial cable having a microwave emitter. The cable can have a first passageway extending through the emitter, a second passageway, and a flexible liner between the first passageway and the second passageway. The liner can encircle the first passageway. The liner can have a lubricious coating.

The system can have a fluid in the second passageway.

Yet a further system for delivering microwave energy to a target biological tissue is disclosed. The system can have a balloon catheter, a coaxial cable in the catheter, a guidewire, and a mechanism to measure properties of the target biological tissue or proximity, wherein the properties are at least one of temperature, magnetic field, electrical conductivity, thermal radiation, and impedance. The coaxial cable can have a microwave emitter. The coaxial cable can have a first passageway extending through the coaxial cable and a second passageway. The system can have a boundary between the first passageway and the second passageway. At least one passageway is in fluid communication with the catheter. The guidewire is in one of the passageways.

The system can have a power source configured to deliver power to the coaxial cable. The system can have transmission lines (e.g., coaxial cables, coaxial connectors, printed circuit boards, etc.) connected to the coaxial cable. These transmission lines can form an impedance transform.

The system can have impedance matching extension transmission lines extending away from the coaxial cable. The impedance matching transmission lines can form a quarter wave transform with either the microwave energy source (e.g., microwave generator) or load microwave antenna) or both.

The system can have a microwave receiver. For example the microwave emitter can be used as a receiver.

Further disclosed is a method for delivering microwave energy to a target biological tissue. The method can include positioning a guidewire adjacent to the target biological tissue. The method can include delivering a coaxial cable over the guidewire. The cable can have a microwave emitter. The cable can have a cable longitudinal axis. The cable can have an inner conductor, an outer conductor insulated from and radially outside of the inner conductor, and a lumen radially inside of the outer conductor. The lumen can extend through the emitter, and the guidewire can slide through the lumen. The method can include removing the guidewire from the lumen. The method can include delivering a fluid to the lumen. The fluid can flow in the first lumen longitudinally distal to the emitter.

The method can include that after the fluid is flowing in the first lumen, the fluid can then flow radially outside of the emitter, and then flow in a fluid passageway proximal to the emitter.

The delivery of the fluid can occur after the removal of the guidewire.

An additional method for delivering microwave energy to a target biological tissue is disclosed. The method can include delivering a coaxial cable adjacent to the target biological tissue. The cable can have a microwave emitter, an inner conductor, an outer conductor insulated from and radially outside of the inner conductor, and a first passageway extending through the coaxial cable. The first passageway can have a port distal to the emitter. The method can include delivering a fluid through the first passageway and the port. The method can include transference of microwave energy from the antenna to the target biological tissue, and wherein the delivery of the fluid occurs concurrently with the transference of energy.

The method can include occluding the end of the first passageway distal to the emitter. The occluding can include closing the first passageway fluid-tight.

A balloon can be in fluid communication with the first passageway. The method can include inflating the balloon. The inflating can include selectively positioning the antenna in a biological vessel adjacent to the target biological tissue.

The method can include detecting a temperature of biological tissue at or adjacent to at least one of the target biological tissue, fluid, emitter, coaxial cable, power input connector, or electromagnetic field radiated by the emitter.

The delivery of the fluid can include delivering the fluid at a flow rate, and controlling the flow rate based at least in part on the detected temperature of at least one of the biological tissue, fluid, emitter, coaxial cable, a power input connector, or the electromagnetic field radiated by the emitter.

The method can include inflating the balloon outside of the antenna.

The cable can have a dielectric insulator between the inner conductor and the outer conductor. The first passageway can extend through the dielectric.

The coaxial cable can have a second passageway. The method can include delivering the fluid to the second passageway.

The method can include inserting a guidewire, introducing the fluid, and connecting a power source to a connector at a proximal terminal end of the coaxial cable. The connector can have an impedance matching circuit connecting the power source to the coaxial cable.

Further disclosed is a method for delivering microwave energy to a target biological tissue. The method can include delivering a catheter adjacent to the target biological tissue. The catheter can have a balloon at a distal end of the catheter. The delivery can include positioning the balloon adjacent to the target biological tissue. The method can include delivering a coaxial cable adjacent to the target biological tissue, wherein the coaxial cable is inside the catheter. The cable can have a microwave emitter. The emitter can be positioned adjacent to the target biological tissue. The emitter can have an emitter longitudinal axis. The coaxial cable can have a first passageway radially inside the emitter. The method cart include actively circulating fluid through the passageway, distal to the emitter, radially outside of and longitudinally coincidental with the emitter, and through the catheter proximal to the emitter and the balloon. The fluid can he delivered toward the distal end of the passageway, toward the proximal end of the passageway, or in alternating directions.

Circulating the fluid through the catheter can include flowing the fluid through a second passageway defined between the radial outside of the cable and the radial inside of the catheter. Circulating the fluid through the catheter can include flowing the fluid through a second passageway radially inside the cable.

The method can include delivering fluid out of a distal port at the distal terminal end of the balloon. The method can include actively or passively closing a configuration at the end of the first passageway distal to the antenna. The first passageway can be at least partially surrounded by a liner. The first passageway can be inside of a lumen in an inner conductor. The lumen can be defined by an inner conductor inner wall. The liner can be unsecured to the inner conductor inner wall around the entire radius of the inner conductor inner wall.

The end of the first passageway distal to the emitter can have fluid ports and/or pores proximal to a controllably closable configuration. The method can include closing the first passageway distal to the fluid ports or pores with the controllably closable configuration. The controllably closable configuration can have a valve, an inflatable occluding balloon, or combinations thereof.

The method can include delivering power to the emitter via a power supply, delivering the fluid via a fluid supply, delivering a guidewire through the emitter via a second passageway in the cable, attaching a connector to the proximal end of the cable, connecting the power supply and fluid supply to the connector, and inserting the guidewire through the connector.

### BRIEF DESCRIPTION OF THE DRAWINGS

FIG. 1 illustrates a stripped away view of a variation of a known microware antenna coaxial cable.

FIG. 2a is a bottom view of a variation of the apparatus.

FIGS. 2b and 2b′ are variations of cross-section A-A of FIG. 2a.

FIG. 2c is a variation of cross-section B-B of FIG. 2a.

FIG. 2d is a variation of close-up view E-E of FIG. 2b′.

FIGS. 3a and 3b are side and top perspective views, respectively, or a variation of the apparatus.

FIGS. 4a through 4c are variations of perspective, side, and front views, respectively, of the power input connector.

FIG. 4d is a variation of cross-sectional view H-H of FIG. 4c.

FIG. 5 is a variation of a schematic view of the circuit diagram of the apparatus.

FIG. 6a is a variation of close-up view C-C of FIG. 2b in a configuration with a guidewire.

FIG. 6b is a variation of cross-section G-G of FIG. 6a.

FIG. 7a is a variation of close-up view C-C of FIG. 2b in a configuration with flow through the fluid inlet.

FIGS. 7b and 7c are variations of cross-section H-H of FIG. 7a.

FIG. 8a is a variation of close-up view D-D of FIG. 2b.

FIGS. 8b through 8h are variations of cross-section J-J of FIG. 8a.

FIG. 9a is a variation of close-up view D-D of FIG. 2b with the balloon in a deflated configuration.

FIG. 9b is a variation of cross-section K-K of FIG. 9a.

FIG. 10a is a variation of FIG. 9a with the balloon in an inflated configuration.

FIG. 10b is a variation of cross-section K-K of FIG. 10a.

FIG. 10a′ is a variation of close-up view D-D of FIG. 2b with the balloon in a deflated configuration.

FIGS. 10b′ is a variation of FIG. 10b with the balloon in an inflated configuration.

FIG. 11 is a variation of close-up view D-D of FIG. 2b with the balloon in an inflated configuration.

FIG. 12a is a variation of close-up view D-D of FIG. 2b with the balloon in a deflated configuration.

FIG. 12b is a variation of FIG. 12a with the balloon in inflated configuration.

FIG. 13a is a variation of cross-section K-K with the distal ends of the fluid delivery passageways in open configurations.

FIG. 13a′ is a perspective view of a variation of a length of the cable at cross-section K-K of FIG. 13a.

FIG. 13b is a variation of FIG. 13a with the distal ends of the fluid delivery passageways in closed configurations.

FIG. 13b′ a perspective view of a variation of a length of the cable at cross-section K-K of FIG. 13b.

FIG. 14a is a variation of cross-sectional view E-E with the distal ends of the fluid delivery passageway in an open configuration.

FIG. 14b is a variation of FIG. 14a with the distal ends of the fluid delivery passageway in a closed configuration.

FIG. 15a is a variation of cross-sectional view D-D of FIG. 2d with the distal ends of the fluid delivery passageway in an open configuration.

FIG. 15b is a variation of FIG. 15a with the distal ends of the fluid delivery passageway in a closed configuration.

FIG. 16a illustrates a variation of the valve and associated elements of FIGS. 15a and 15b when the valve is in a closed configuration.

FIG. 16b illustrates a variation of the valve and associated elements of FIGS. 15a and 15b when the valve is in a closed configuration.

FIG. 17a illustrates a variation of cross-sectional view L-L of FIGS. 15a and 15b when the valve is in an opened configuration.

FIG. 17b illustrates a variation of cross-sectional view L-L of FIGS. 15a and 15b when the valve is in a closed configuration.

FIG. 18a is a variation of a side view of the distal terminal end of the apparatus, with the catheter and balloon not shown for illustrative purposes.

FIGS. 18b and 18c are variations of cross-sectional view M-M of FIG. 18a.

FIG. 19a is a variation of close-up view D-D of FIG. 2d with the delivery fluid passageway in an opened configuration, with the catheter and balloon not shown for illustrative purposes.

FIG. 19b is a variation of FIG. 19a with the delivery fluid passageway in a closed configuration, with the catheter and balloon not shown for illustrative purposes.

FIG. 20a is a variation of close-up view D-D of FIG. 2d with the delivery fluid passageway in an opened configuration, with the catheter and balloon not shown for illustrative purposes.

FIG. 20b is a variation of FIG. 20a with the delivery fluid passageway in a closed configuration, with the catheter and balloon not shown for illustrative purposes.

FIG. 21a is a variation of close-up view D-D of FIG. 2b with the balloon in a deflated configuration.

FIG. 21b is a variation of FIG. 21a with the balloon in an inflated configuration.

FIG. 22a is a variation of close-up view D-D of FIG. 2d with the delivery fluid passageway in an opened configuration, with the catheter and balloon not shown for illustrative purposes.

FIG. 22b is a variation of FIG. 22a with the delivery fluid passageway in a closed configuration, with the catheter and balloon not shown for illustrative purposes.

FIG. 23a is a variation of cross-sectional view K-K with the delivery fluid passageway in an opened configuration, with the catheter and balloon not shown for illustrative purposes.

FIG. 23b is a variation of FIG. 23a with the delivery fluid passageway in a closed configuration, with the catheter and balloon not shown for illustrative purposes.

FIG. 24 is a variation of close-up view D-D of FIG. 2d.

FIG. 25a is a variation of close-up view D-D of FIG. 2d with the delivery fluid passageway in an opened configuration, with the catheter and balloon not shown for illustrative purposes.

FIG. 25b is a variation of FIG. 25a with the delivery fluid passageway in a closed configuration, with the catheter and balloon not shown for illustrative purposes.

FIGS. 26a and 26b are partially see-through views of variations of the distal terminal end of the apparatus.

FIG. 27 is a variation of a simplified lateral cross-section of the guidewire passageway and the central lumen.

FIG. 28 illustrates a variation of a method for using the apparatus.

FIG. 29 is a close-up view of a variation of a distal end of the apparatus with a see-through view of the balloon for illustrative purposes.

FIGS. 30a through 30c are perspective, side, and distal end views, respectively of a variation of the distal can of the apparatus including the balloon. FIG. 30c further shows a variation of using the apparatus in an exemplary vessel wall.

FIG. 31 illustrates a variation of the distal end of the apparatus including the balloon.

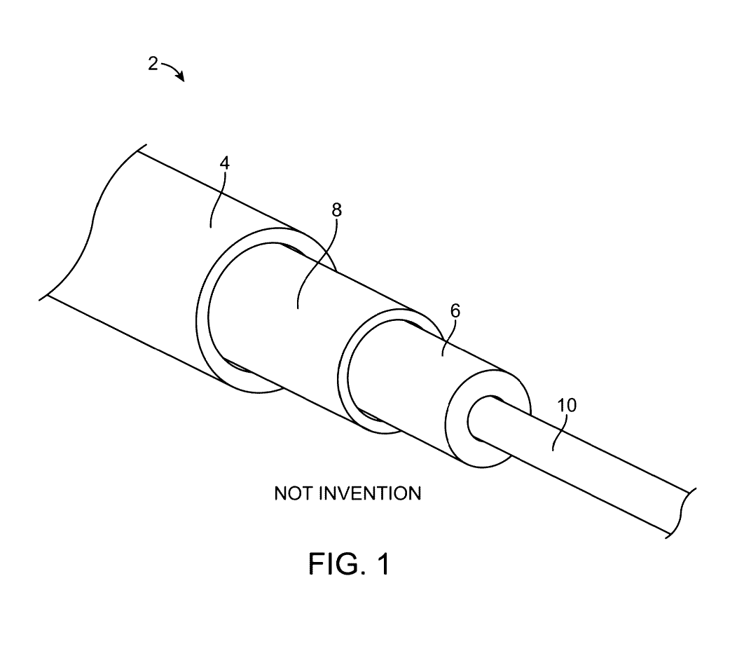

### DETAILED DESCRIPTION

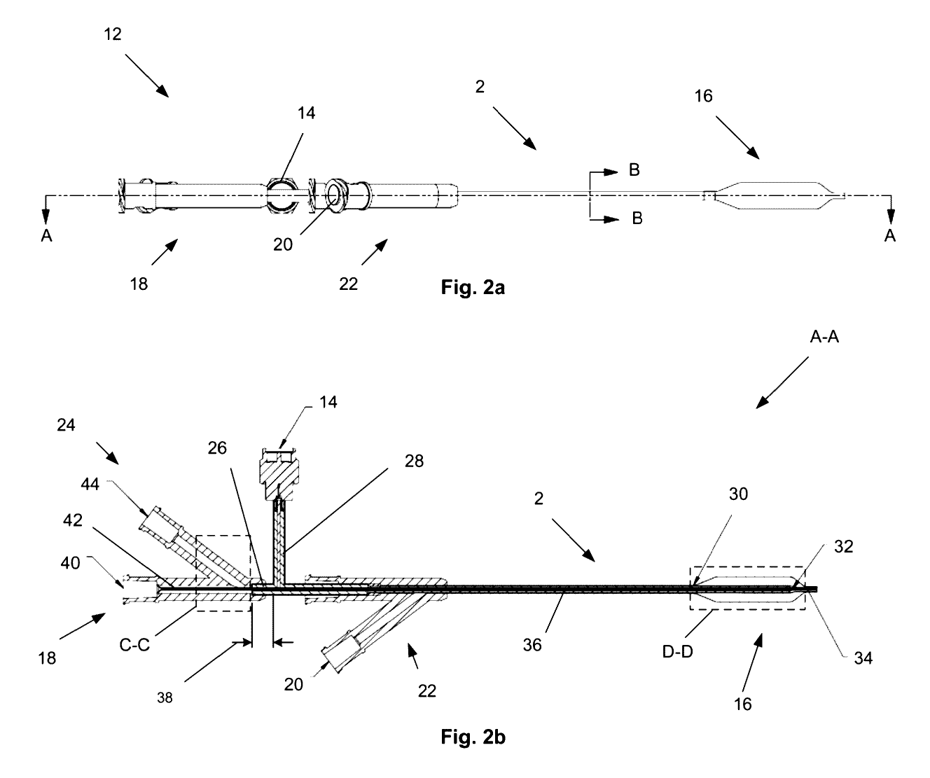

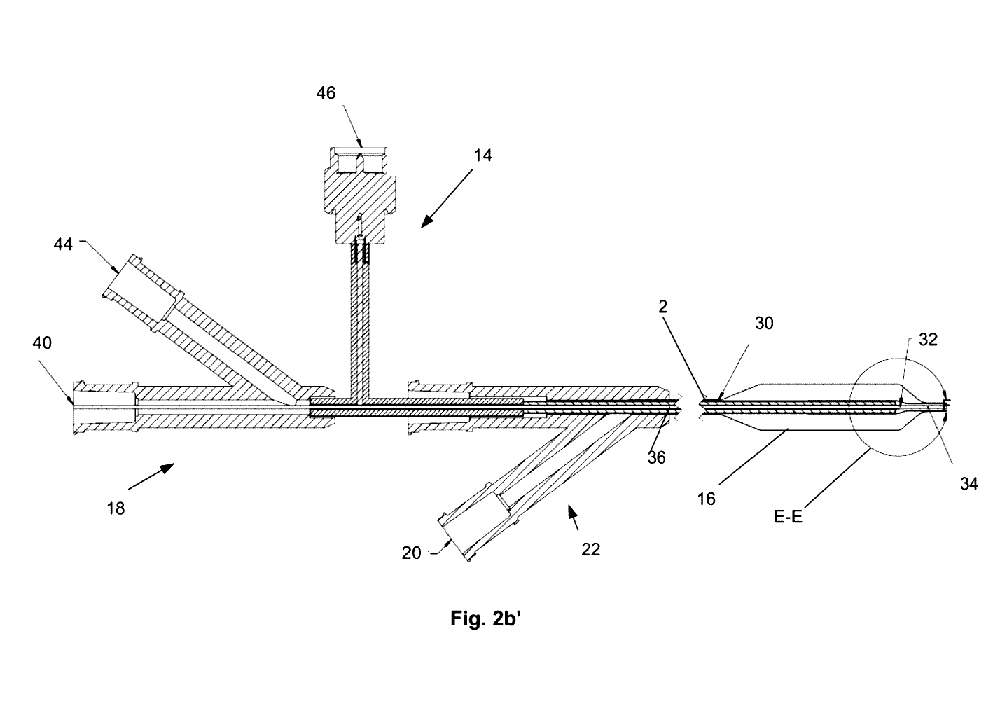

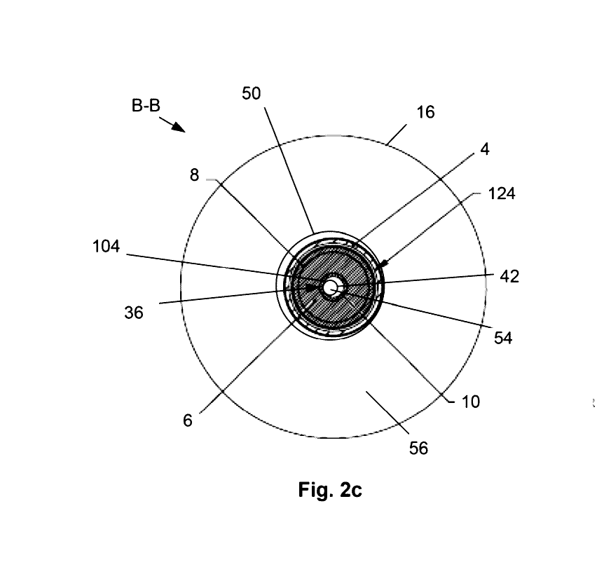

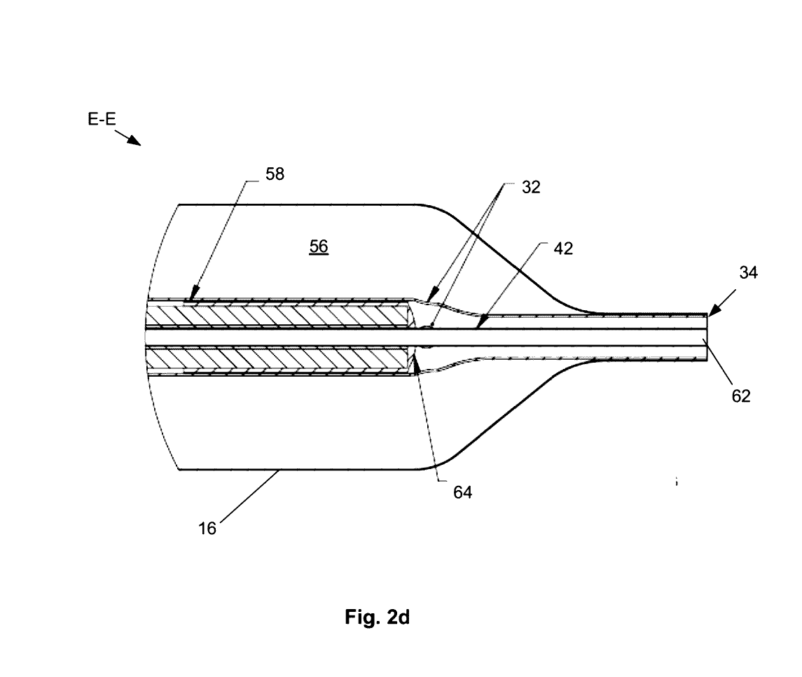

FIG. 2a through 2d illustrate that a microwave antenna cable system 68 or apparatus 12 can have a cable 2 with a balloon 16 at the distal end of the cable 2. The cable system 68 can be used to deliver microwave energy to a microwave emitter, such as one or more antennae 58, within the balloon 16. The balloon 16 can be positioned in a body lumen with a body lumen wall, and the balloon 16 can be inflated near or in contact with the body lumen wall (e.g., a blood vessel wall 256). A fluid (e.g., liquid saline solution, water, carbon dioxide, or combinations thereof) can be circulated through the balloon 16, for example to decrease the thermal energy delivered through the balloon 16, decrease or increase the temperature of the components within the balloon 16, such as the antenna 58, increase the force delivered by the balloon 16 to the exterior environment, or combinations thereof.

The cable system 68 can have a connector system 70 having one or more elements configured to attach to and detach from separate inputs and outputs for matter (e.g., fluid), energy, one or more tools, data, or combinations thereof. For example, the connector system 70 can have a separate fluid input connector 18, fluid output connector 22, and a power input connector 14. The cable 2 can have one or more inner lumens 36. The inner lumens 36 can have one or more passageways. The passageways can be configured to allow for the flow of fluid and/or movement of solids (e.g., guidewires, other tools) to and/or from the balloon and/or out of or into the distal end of the balloon.

The fluid input connector 18 can be attached to the proximal terminal end of the cable 2 and/or to the proximal terminal end of the power input connector 14. The fluid input connector 18 can have or be a three-way connector 24, such as a T-connector or Y-connector. The fluid input connector 18 can have a guide wire port 62 configured to receive a guidewire 102 and/or other mechanical tools, and a separate flow inlet 44. The flow inlet 44 can be configured to attach to a pressurized fluid source. The flow inlet 44 and guidewire port 40 can converge and merge. The guidewire port 40 can have central lumen having a lubricious liner 42. During use, the guidewire 102 can be in contact with a lubricious surface of the lubricious liner 42.

The fluid outflow connector can be attached to the proximal terminal end of the cable 2, for example distal to the distal end of the fluid input connector 18. The fluid output connector 22 can have a fluid outlet extending away from the cable 2. The fluid outlet can be configured to attach to a reservoir and/or a suction source.

The power input connector 14 can be attached to the proximal terminal end of the cable 2 and/or to the fluid output connector 22, for example seated inside of the proximal half of the fluid output connector 22. The power input connector 14 can have a power input extension 28 extending perpendicularly away from the cable longitudinal axis. The end of the power input extension 28 away from the cable longitudinal axis can be a power input 46 and attach to a power source 94, such as a microwave generator (e.g., having a traveling-wave tube (TWT) such as a Klystron and/or magnetron), either via direct attachment or another transmission line such as a coaxial cable.

The power input connector 14 can incorporate an impedance matching section either as an extension to or as pan of the power input connector 14. The impedance matching section of the power input connector 14 can be ¼ of the wavelength of the frequency of the emitted microwave energy, for example to ensure efficient transfer of power from the microwave source to the coaxial cable.

The distal end of the inner lumen 36 and/or an inner flow passageway 104 can have a delivery tip valve 34. The tip valve 34 can have a fluid-tight seal the distal end of the balloon 16.

The cable 2 can have one or more return flow passageways 114. The distal ends of the return flow passageways 114 can terminate at return flow passageway ports 30 within the balloon 16, for example at the proximal terminal end of the balloon 16. The return flow passageways 114 can proximally terminate at the fluid output connector 22, for example in fluid communication with the flow outlet 20. The return flow passageway 114 can extend through the cable 2, for example radially outside of the inner lumen 36.

The cable 2 can have one or more inner flow passageways 104. The inner flow passageway's 104 can extend through the inner lumen 36. The distal ends of the inner flow passageways 104 can terminate at inner flow passageway ports 32 within the balloon 16, for example at the distal to the return flow passageways ports 30 and emitter. The return flow passageways 114 can proximally terminate at the fluid input connector 18, for example in fluid communication with the flow inlet 44.

FIG. 2c illustrates that the inner lumen 36 can extend along cable longitudinal axis at the radial center of the cable 2. The lubricious liner 42 can divide the inner flow passageway 104 from the guidewire passageway 54 in the inner lumen 36. The lubricious liner 42 can be lubricious on one or both surfaces. The lubricious liner 42 can have a lower coefficient of friction compared with the coefficient of friction of the inner surface of the inner lumen 36, for example when both surfaces are wet or dry.

The cable 2 can have an inner conductor .10 in contact with and radially outside of the inner lumen 36. The lubricious liner 42 can be cylindrical and connect to the inner surface of the inner lumen 36 along a single solid or broken/dashed line parallel with the cable longitudinal axis.

The inner conductor 10 can be in contact with and radially inside of a dielectric insulator 6. The dielectric insulator 6 can be in contact with and radially inside of an outer conductor 8. The outer conductor 8 can be in contact with and radially inside of a cable jacket 4. The cable jacket 4 can be an electrical insulator. The cable jacket 4 can be in contact with and fixed to, or spaced away and slidable within a catheter 50. For example, the return flow passageways 114 can be between the cable jacket 4 and the catheter 50 or in the cable jacket 4.

In an inflated configuration 260, the balloon 16 can have a larger maximum radius than the catheter 50. In an inflated configuration 260, the balloon 16 can define a balloon reservoir 56 volume filled with fluid within the balloon 16.

FIG. 2d illustrates that the lubricious liner 42 can extend beyond the distal terminal end of the distal-most antenna 58. The inner flow passageway 104 can have one or more radial layers of inner flow passageway ports 32 to deliver fluid from the inner flow passageway 104 to the balloon reservoir 56 or volume.

The inner conductor 10 can be soldered to the power input connector 14 inner conductor 10. The outer conductor 8 can be soldered to the power input connector 14 outer conductor 8. The inner conductor 10 can be fixed, joined, or otherwise attached to the antenna tip 246 at an inner conductor 10 joint. For example, the inner conductor 10 joint can be a soldered joint 64,

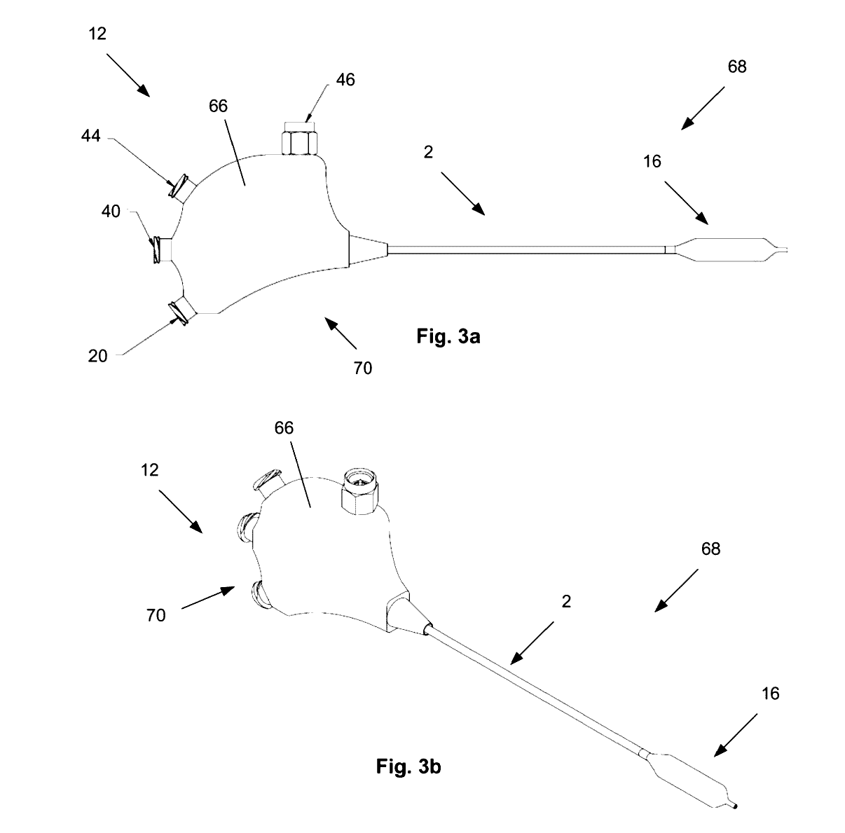

FIGS. 3a and 3b illustrate that the apparatus 12 can have a connector system 70 having a single case or handle 66 with the flow outlet 20, guidewire port 40, flow inlet 44, and power input 46. The flow outlet 20, guidewire port 40, flow inlet 44, and power input 46 can be coplanar. The cable 2 can be coplanar with the flow outlet 20, guidewire port 40, flow inlet 44, and power input 46.

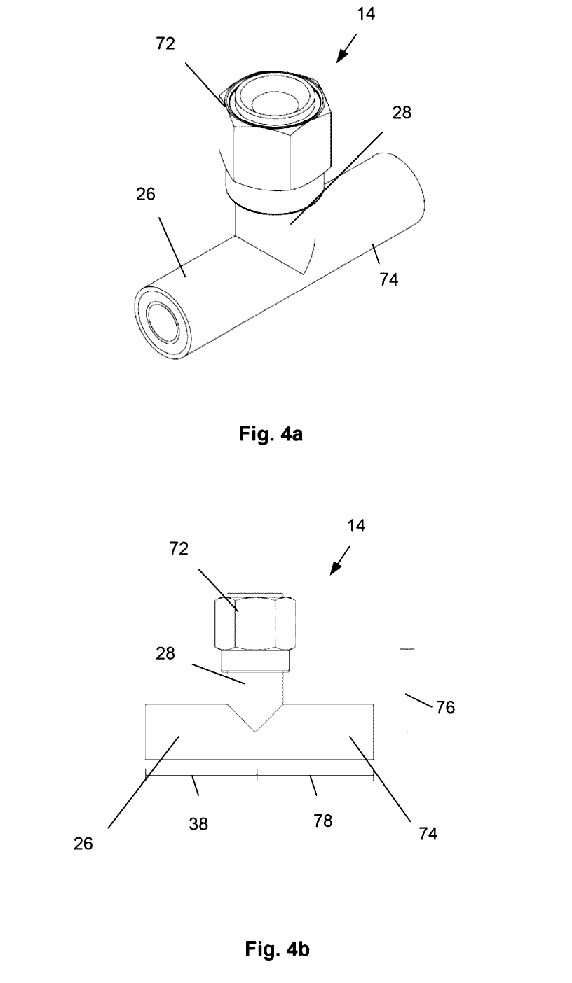

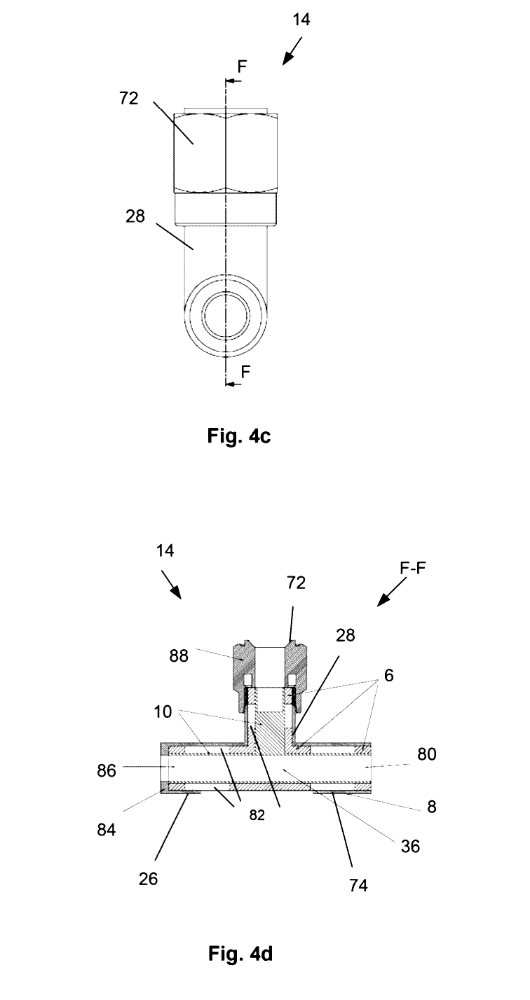

FIGS. 4a through 4d illustrate that the power input connector 14 can have a T-type configuration. Power input connector 14 can have an extension that can terminate at a power source coupler 72 configured to create a detachable connection to a power source input, such as a 2.45 GHz or 5 GHz power source 94.

The power input connector 14 can have a distal extension 74 extending distally from the juncture of the power input extension 28 and impedance matching extension 26. The impedance matching extension 26 can extend proximally from the juncture. The power input extension 28 can extend perpendicularly from the longitudinal axes of the impedance matching extension 26 and/or distal extension 74. The impedance matching extension 26 longitudinal axis can be collinear with distal extension 74 longitudinal axis.

FIG. 4c illustrates that the power input extension 28 can have a power input extension length 76. The distal extension 74 can have a distal extension length 78. The power input extension length 76, distal extension length 78, and impedance matching extension length 38 can be equal to each other. For example, the power input extension length 76, distal extension length 78, and impedance matching extension length 38 can be about ⅛ of the wavelength of the input power.

The power input extension 28 to the impedance matching extension 26 can be an impedance transform. The transform can create a ¼ wave transform, for example for impedance matching. the end of this transform, the outer connector can be short-circuited to the inner connector. The short circuit and ¼ transform can make this transform perform as an open circuit as seen from the power input 46 connected to the power source coupler 72. This transform can, for example, prevent energy from the power source 94 from traveling through the impedance matching extension 26 and radiating out of the proximal end of the power input connector 14.

The power source coupler 72 to the distal extension 74 can be an impedance transform. This transform can transforms the impedance of the cable 2 to match the impedance of the power source 94 for maximum power transfer to the cable 2 from the source.

An impedance transform can be a length of transmission line (e.g., coaxial cable or traces on a PCB) that can allow the transformation of a source impedance to a load impedance for a particular frequency or a range of frequencies. Impedance transformations can be used to either match a source to a load to allow optimal power transfer or to block power from going to a certain target. The impedance matching extension 26 can have the length of a quarter wave transform (i.e., the length of the impedance matching extension 26 can be a quarter wavelength long at the operating frequency of the input power) as measured from the power input extension 28. This transform can be terminated at the proximal terminal end of the impedance matching extension 26 in an outer conductor to inner conductor short-circuit 84. The quarter-wave transform can effectively make the impedance matching extension 26 act as an open circuit, for example, preventing, or minimizing, energy loss caused by signal reflections, conduction or radiation from the impedance matching extension 26 which can otherwise interfere (i.e., destructively reduce) power delivery through the distal extension 74 to the cable 2.

FIG. 4d illustrates that the inner conductor 10, outer conductor 8, and dielectric insulator 6 can extend through the power input connector 14. The inner conductor 10, outer conductor 8, and dielectric insulator 6 can extend perpendicular to the longitudinal axis of the cable 2, along the power input extension 28.

The inner lumen 36 can extend through the power input connector 14. The power input connector 14 inner lumen 36 can have a power input connector inner lumen inlet 86 at the proximal terminal end of the impedance matching section, for example to receive the guidewire 102 and fluid, and a power input connector inner lumen outlet 80 at the distal terminal end of the distal extension 74, for example through which the guidewire 102 can extend and fluid can flow distally through the cable 2.

The power source coupler 72 can have a female or male coaxial connector power entry 68.

The dielectric insulator 6 can have PTFE and/or air gaps 82.

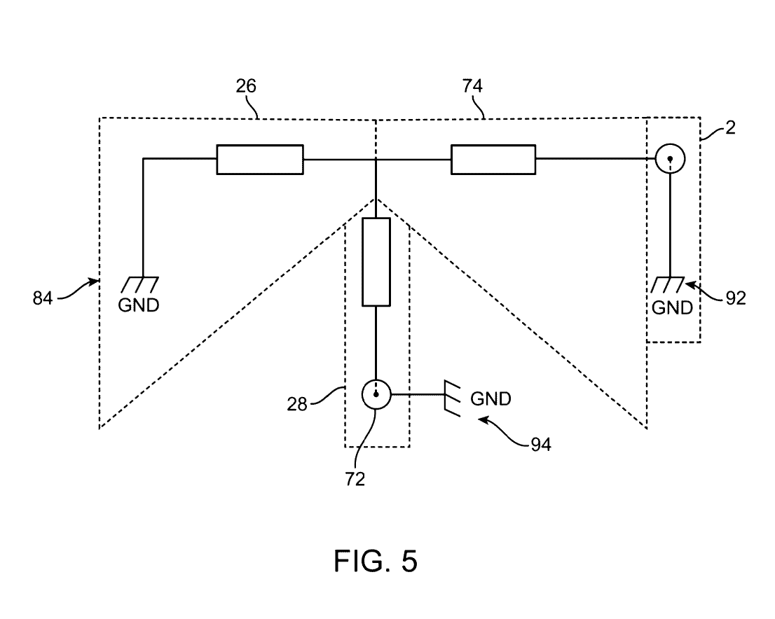

FIG. 5 illustrates that the impedance matching extension 26 can make a quarter wave transform from the power source. For example, the power input extension 28 can have a transmission line length of ⅛ of the wavelength of the input power. The impedance matching extension 26 can have a transmission line length of ⅛ of the wavelength of the input power and be terminated in a short circuit between the inner and outer conductors 8.

The distal extension 74 can have a transmission line length such that the microwave source impedance and the cable 2 impedance are perfectly matched.

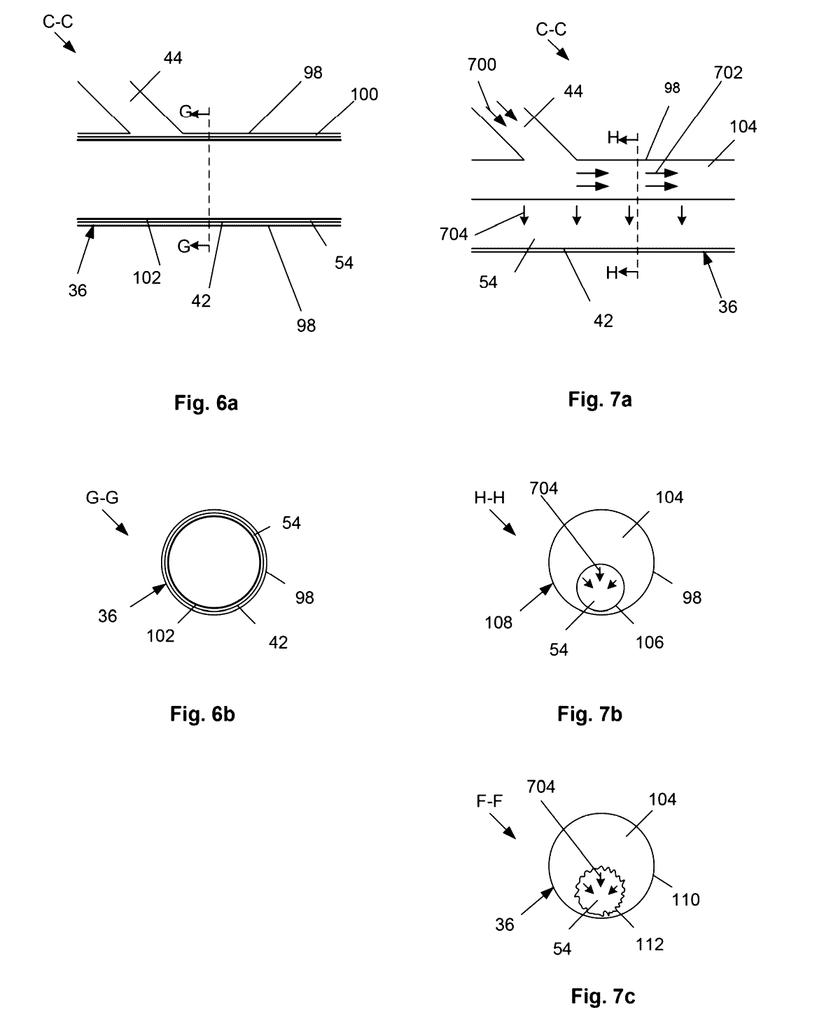

FIGS. 6a and 6b illustrate that the fluid input connector 18 can have a fluid connector inner wall 98 defining the inner lumen 36 in the fluid input connector 18 (e.g., in a T-connector or Y-connector). A guidewire 102 can be inserted through the inner lumen 36, for example in the cylindrical lubricious liner 42 in the guidewire passageway 54. The lubricious liner 42 can be made from or the inner surface can be coated with a low-friction material, such as PTFE, and/or a wetting agent. The guidewire 102 can substantially completely occlude (i.e., fill) the inner lumen 36, for example the guidewire diameter can be about the diameter of the inner lumen 36 in combination with the thickness of the lubricious liner 42.

The flow inlet 44 can be in fluid communication with the inner lumen 36. When the guidewire 102 is in the inner lumen 36 extending across the intersection of the flow inlet 44 with the inner lumen 36, the guidewire 102 can obstruct the flow inlet 44, preventing flow from the flow inlet 44 to the inner lumen 36.

FIGS. 7a through 7c illustrate that the guidewire 102 can be retracted and removed from the inner lumen 36 of the fluid input connector 18. Fluid can then be delivered through the flow inlet 44, as shown by arrows 700. The fluid pressure from the fluid entering from the flow inlet 44 and flowing along an inner flow passageway 104, as shown by arrows 702, can deliver pressure to push the lubricious liner 42 away from at least one side of the fluid input connector wall, as shown by arrows 704, compressing or contracting the liner wall and opening the inner flow passageway 104 or channel. The lubricious liner 42 can radially contract elastically 106 (shown in FIG. 7b) or inelastically 112 (shown in FIG. 7c), as shown by arrows 704. The inner flow passageway 104 can be formed between the lubricious liner 42 and the inner lumen 108.

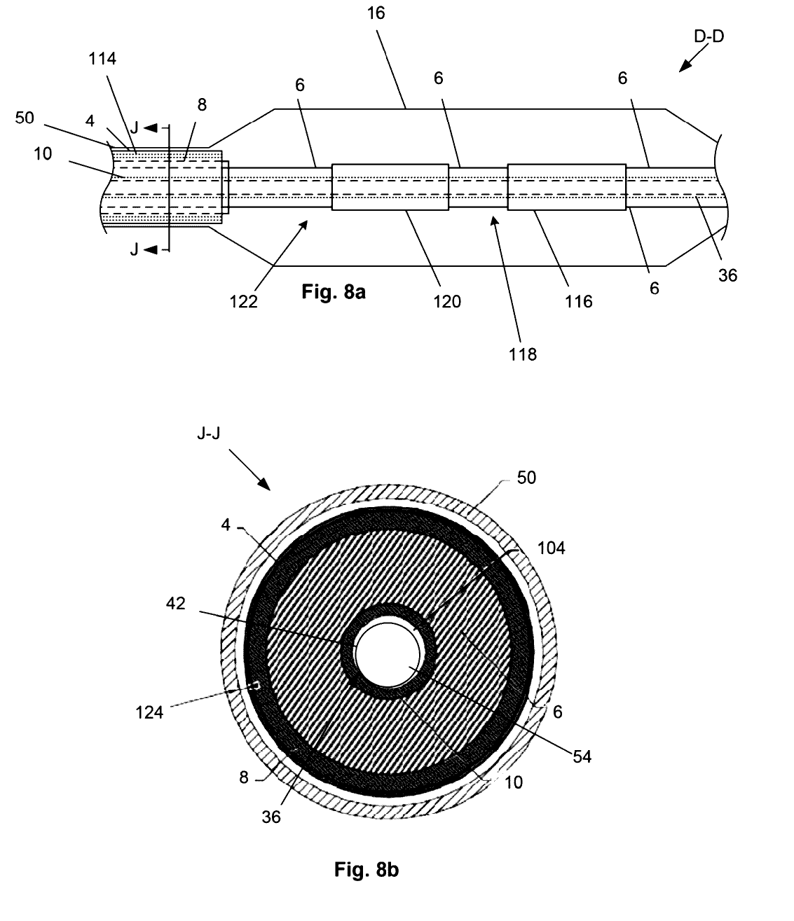

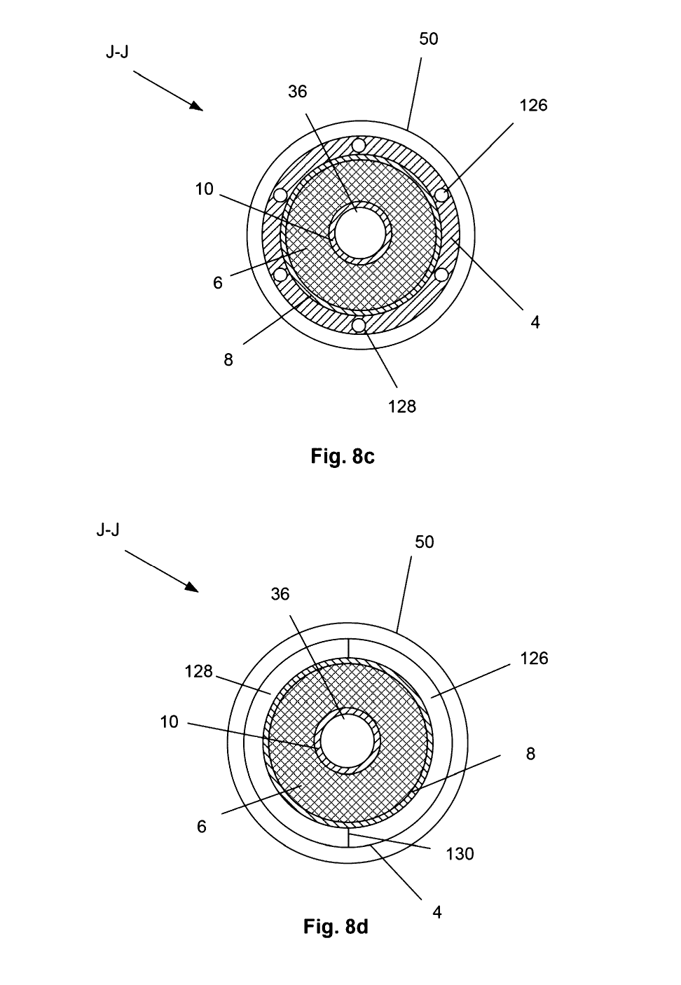

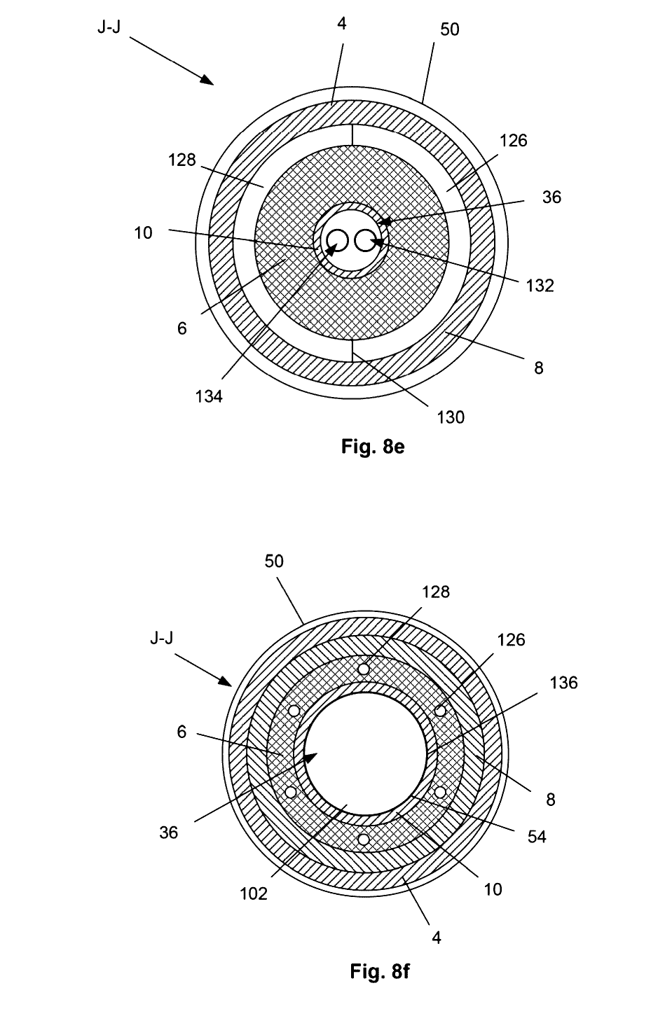

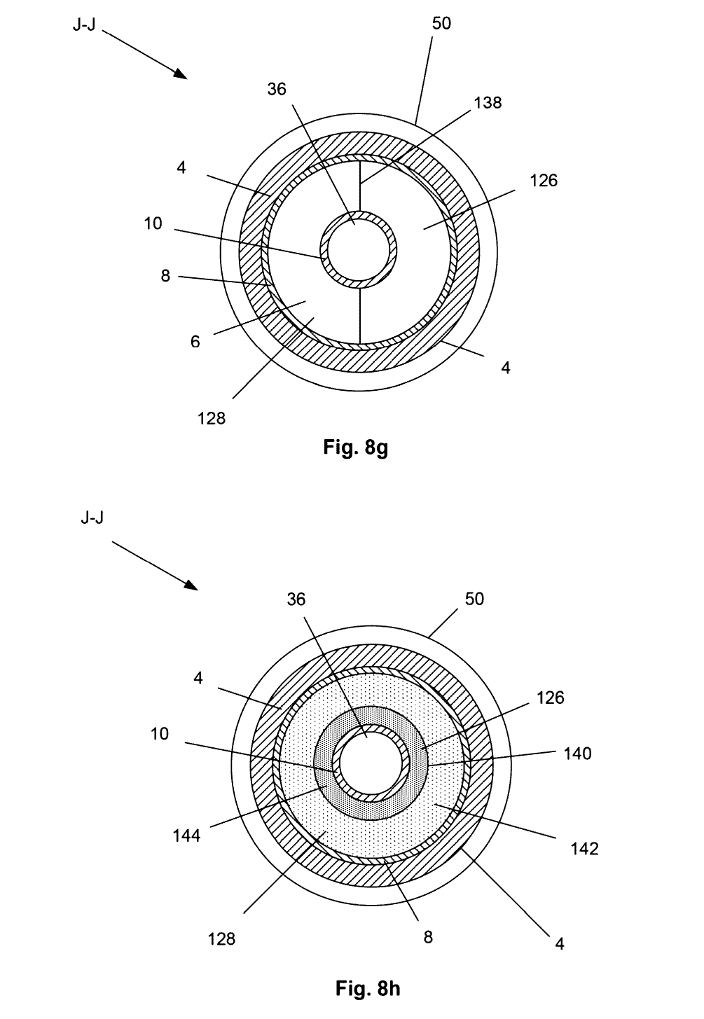

FIG. 8a illustrates that the emitter 92 can have a first antenna 58, such as a metal spacer 120, and a second antenna 58, such as a distal antenna tip 116. The emitter 92 can have a first slot 118 between the first antenna 58 and a second antenna 58, and a second slot 122 or gap between the first antenna 58 and the distal terminal end of the outer conductor 8.

FIGS. 8a and 8b illustrate that the return or outer flow passageway 124 can be radially between the catheter 50 and the cable jacket 4. The outer flow passageway 124 can be cylindrical and coaxial with the cable longitudinal axis.

FIG. 8c illustrates that the delivery and/or return flow passageways 114 can be in (i.e., within the radial limits of) the cable jacket 4. For example, the flow passageways can he cylindrical. The flow passageway diameters in the cable jacket 4 can have diameters less than the thickness of the cable jacket 4. The cable 2 can have three delivery flow passageways 154 and three return flow passageways 114. The delivery flow passageways 154 can alternate angularly with the return flow passageways 114. For example, first outer flow passageways 126 can be for return flow 178, and second outer flow passageways 128 can be for delivery flow. The first 126 and second 128 outer flow passageways 124 can have flow in the same direction, opposite directions, or alternate during use.

FIG. 8d illustrates that the flow passageways can have semi-cylindrical flow passageways. The angularly adjacent flow passageways can have the same or alternate flow directions. The cable 2 can have radially-extending walls or dual-lumen extrusions 130, including load-bearing cross-braces and/or non -load-bearing walls, between the outer conductor 8 and the cable jacket 4. The radially-extending walls can form dividers between the adjacent flow passageways. The flow passageways can be between the cable jacket 4 and the outer conductor 8.

FIG. 8e illustrates that the radially-extending walls can extend from the dielectric. insulator 6 to the outer conductor 8. The outer passageways formed by the radially-extending walls can be between the dielectric insulator 6 and the outer conductor 8.

The cable 2 can have a first inner passageway 134 and a second inner passageway 132 within the first lumen 136. The inner passageways can be cylindrical. The longitudinal axes of the inner passageways can be symmetric with respect to the cable longitudinal axis. The first and second cylindrical passageways can have longitudinal axes parallel with the longitudinal axis of the cable longitudinal axis. The first inner passageway 134 and second inner passageway 132 can be defined respectively by a first inner liner and a second inner liner.

The passageways can be used for any combination of insertion or deployment into the balloon 16 or target tissue site of the guidewire 102, surgical tools, contrast media, therapeutic media, anesthetic media, inflation media, drainage such as suction, and combinations thereof.

For example, the guidewire 102 can be inserted through the first inner passageway 134. One or more surgical tools, contrast media, therapeutic media, anesthetic media, or combinations thereof can be inserted through the second inner passageway 132. The first outer flow passageway 126 can be used for suction and drainage from the balloon 16. The second outer flow passageway 128 can be used to deliver pressurized inflation media to the balloon 16.

FIG. 8f illustrates that the delivery and/or return flow passageways 114 can be in (i.e., within the radial limits of) the dielectric insulator 6. For example, the flow passageways can be cylindrical. The flow passageway diameters in the cable jacket 4 can have diameters less than the thickness of the dielectric insulator 6. The cable 2 can have three delivery flow passageways 154 and three return flow passageways 114. The delivery flow passageways 154 can alternate angularly with the return flow passageways 114.

FIG. 8g illustrates that the dielectric insulator 6 can be angularly divided into the return and delivery outer flow passageways 124 by a dielectric divider, such as radially extending walls 138 between the inner conductor 10 and the outer conductor 8. The dielectric insulator 6 sections can be filled with an insulating material capable of allowing fluid flow in the longitudinal direction, for example sponge, a capillary or wicking fabric, or combinations thereof

FIG. 8h illustrates that the dielectric insulator 6 can be divided into a radially-divided flow passageways, such as a radially inner dielectric insulator 144 and a radially outer dielectric insulator 142, for example divided by a cylindrical dielectric layer divider 140. The radially inner and outer dielectric insulators 142 can be filled with insulating material capable of allowing fluid flow in the longitudinal direction, for example sponge, a capillary or wicking fabric, or combinations thereof. For example, the radially inner insulator can be the first outer flow passageway 126 For example, the radially outer insulator can be the second outer flow passageway 128.

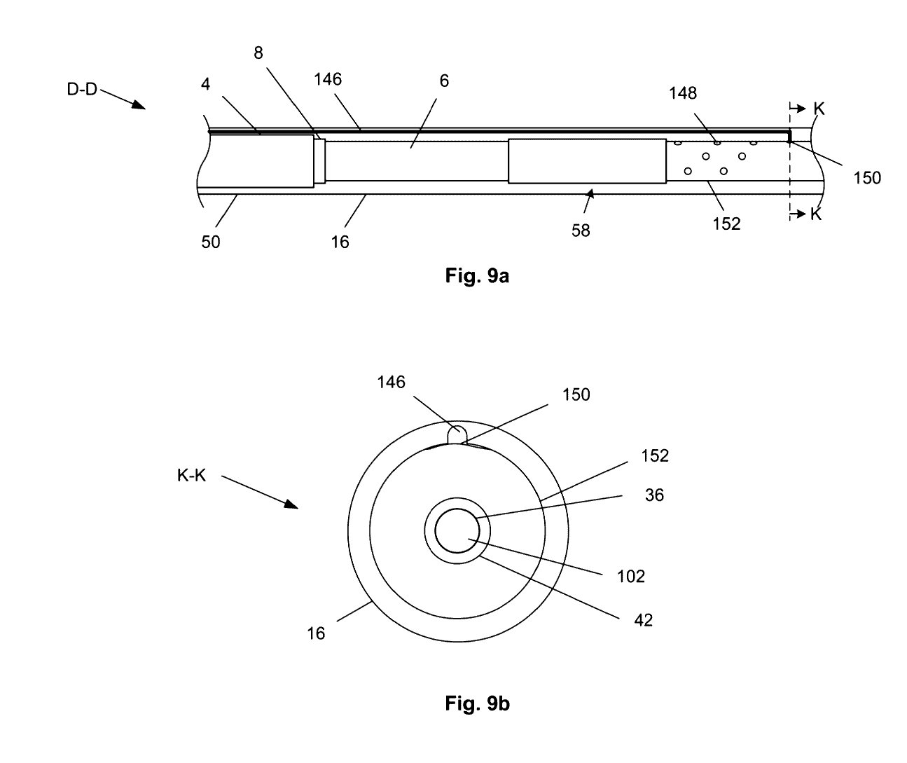

FIGS. 9a and 9b illustrate that the apparatus 12 can have fluid ports 148 in the lateral or radial wall of a lubricious liner 42 or other inner liner, such as the distal extension 74 of the cable jacket 4 or outer wall 110 of the dielectric insulator 6, around a delivery flow passageway 154. In some variations the apparatus 12 can have a lubricious liner 42 with fluid ports 148 and no inner liner radially outside of the lubricious liner 42. The fluid ports 148 can extend through the outer wall 110 of the dielectric insulator 6. The fluid ports 148 can be distal to at least one of the antennae 58 or the entire emitter 92. The fluid ports 148 can open fluid communication between the delivery or inlet flow passageways and the balloon reservoir 56 as well as the return or outlet flow outer or inner passageways or channels. While a distal terminal end of the of the delivery passageway is open, fluid flowing through the delivery flow passageways 154 can largely or entirely flow out of the distal terminal end of the delivery passageways (e.g., into the target site, such as a biological lumen, for example a blood vessel) with no or minimal flow out of the fluid ports 148. The external balloon 214 is shown in a deflated configuration.

The apparatus 12 can have an inflatable bladder 150 or internal balloon attached to the inner liner 152 or radially outside of the inner liner 152. The inflatable bladder 150 can be longitudinally distal to fluid ports 148. The apparatus 12 can have a bladder inflation channel 146 extending from a controllable proximal inflation fluid source distally to the inflatable bladder 150. The inflation channel 212 can be a tube that is not inflatable at pressures equal to or less than the pressure delivered by the proximal inflation source. The proximal end of the inflation channel 212 can have a thinned wall compared to the rest of the inflation channel 212. The thinned wall that can have a failure pressure less than the failure pressure of the inflatable bladder 150. For example when the pressure delivered by the proximal inflation source exceeds the failure pressure of the inflation channel 212, proximal end of the inflation channel 212 (e.g., outside of the patient) can burst and release the inflation fluid before the pressure reaches the failure pressure of the inflatable bladder 150. The inflatable bladder 150 can be in an uninflated or retracted configuration when the guidewire 102 extends through the guidewire passageway 54 in the inner lumen 36 beyond the fluid ports 148, such as extending out of the distal end of the guidewire passageway 54.

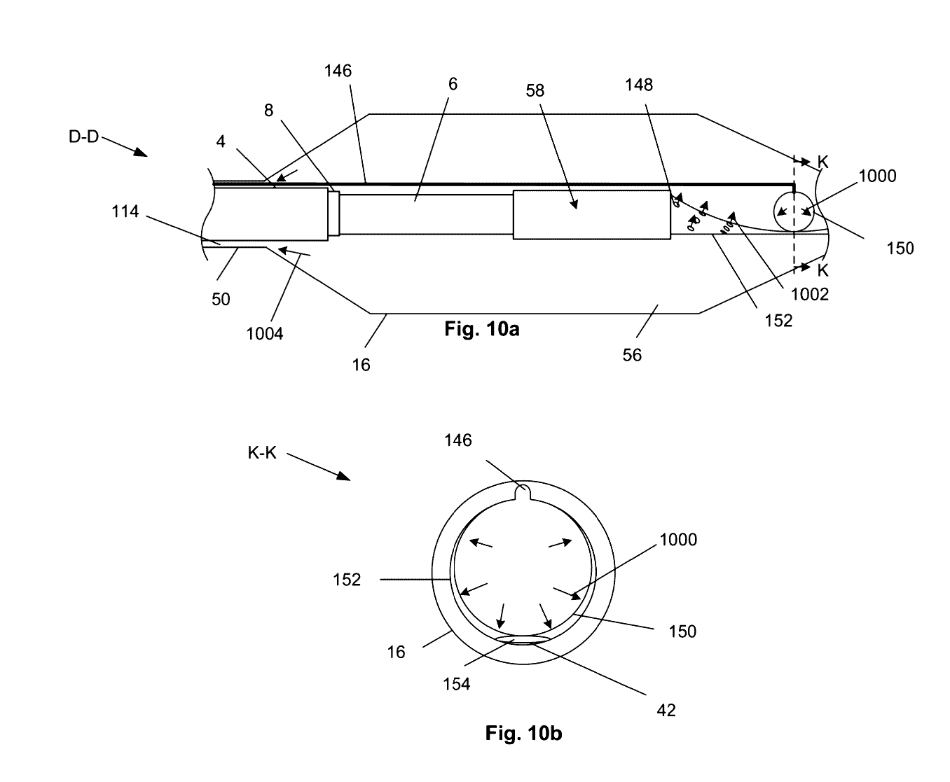

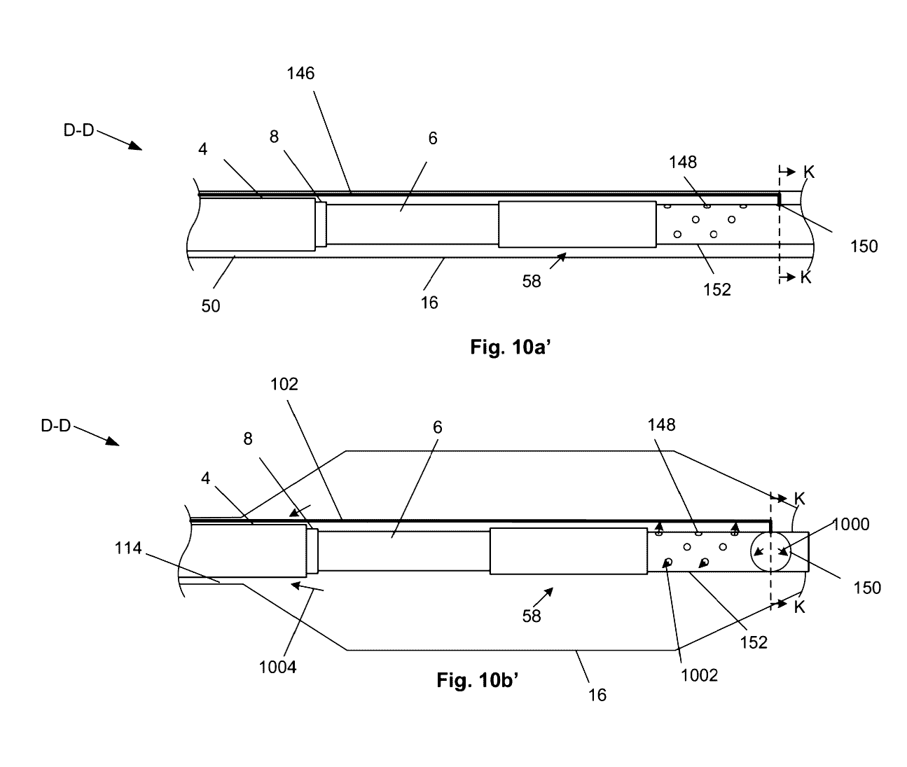

FIGS. 10a and 10b illustrate that the guidewire 102 can be removed from the guidewire passageway 54 in the inner lumen 36. Fluid can then be delivered from the proximal inflation fluid source and flow under pressure through the bladder inflation channel 146 to the inflatable bladder 150. The inflation fluid can then inflate and expand the inflatable bladder 150, as shown by arrows 1000. The inflatable bladder 150 can then pinch, press, collapse, or contract closed the inner liner 152 and/or the lubricious liner 42 proximal of a distal terminal port of the inner liner 152 and/or lubricious liner 42 and distal of the fluid ports 148. The inner liner 152 and lubricious liner 42 can be partially or totally occluded by the wall of the respective liner compressed by the inflated inflatable bladder 150.

Fluid can then flow out of fluid ports 148, as shown by arrows 1002, into balloon reservoir 56. The fluid can then inflate the balloon 16.

The fluid can flow out of the balloon 16 and through the return flow passageway 114, as shown by arrows 1004. The return flow passageway 114 can be between the cable jacket 4 and the catheter 50. Flow can move in either direction: flowing to the balloon 16 through the lubricious liner 42 and inner liner 152 and out of the balloon 16 between the catheter 50 and cable jacket 4 (as shown), or flowing to the balloon 16 between the cable jacket 4 and the catheter 50 and out of the balloon 16 through the lubricious liner 42 and inner liner 152. Flow can oscillate between the flow passageways.

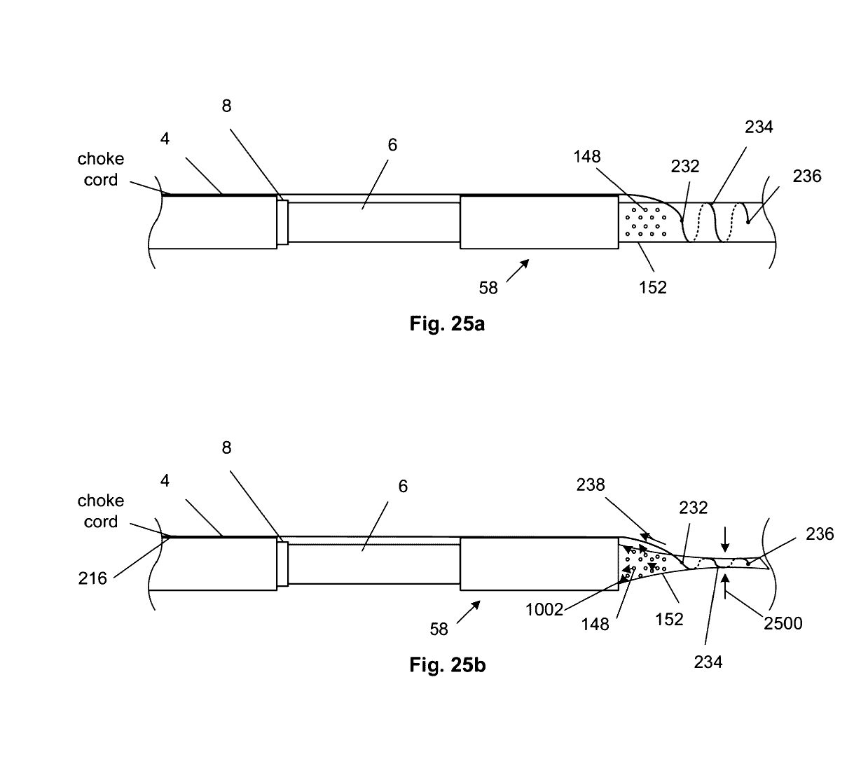

FIG. 10a′ illustrates that the inflatable bladder 150 can be radially inside of the inner liner 152 and/or lubricious liner 42. The inflatable bladder 150 can extend laterally from the radial outside edge of the bladder inflation channel 146. The bladder inflation channel 146 can be adjustably (e.g., by sliding) attached longitudinally to the cable 2. For example, the bladder inflation channel 146 can be a hollow guidewire 102, such as positioned as shown in FIG. 25b.

FIG. 10b′ illustrates that after the guidewire 102 (e.g., a second guidewire if the bladder inflation channel 146 is a first guidewire) is removed from the inner lumen 36, the inflatable bladder 150 can be inflated by inflation fluid flow, as shown by arrows 1000. The inflated inflatable bladder 150 can then partially or totally occlude the inner liner 152 and/or lubricious liner 42, for example forcing fluid delivered inside of the delivery fluid passageways to flow out of the fluid ports 148 and into the balloon 16, as shown by arrows 1002, for example, inflating the balloon 16.

FIG. 11 illustrates that the guidewire 102 can be inserted through the guidewire passageway 54 in the inner lumen 36, or through the fluid passageway in the inner lumen 36, or inserted through the inner lumen 36 having no dividers. The guidewire 102 can have a diameter significantly less than the diameter of the inner lumen 36, for example less than 75%, or more narrowly less than 50% of the diameter of the inner lumen 36. The guidewire 102 can be hollow. The distal terminal end of the guidewire 102 can have an inflatable guidewire tip 156 radially centered about the guidewire 102.

Inflatable fluid pressure can be delivered through a hollow channel in the guidewire 102 to the inflatable guidewire tip 156, for example, inflating the inflatable guidewire tip 156 with inflation fluid flow, as shown by arrows 1000. The inflatable guidewire tip 156 can then occlude the inner fluid passageway. Inflation fluid can then be delivered through the inner lumen 36 around the guidewire 102, out of the fluid ports 148, as shown by arrows 1002, and into the balloon 16, for example inflating the balloon 16.

The guidewire 102 can be a standard guidewire 102 used to guide the system through a lumen during deployment; or a device not used to guide the system during deployment through a lumen, but for example used to occlude the guidewire passageway 54 and/or inner lumen 36.

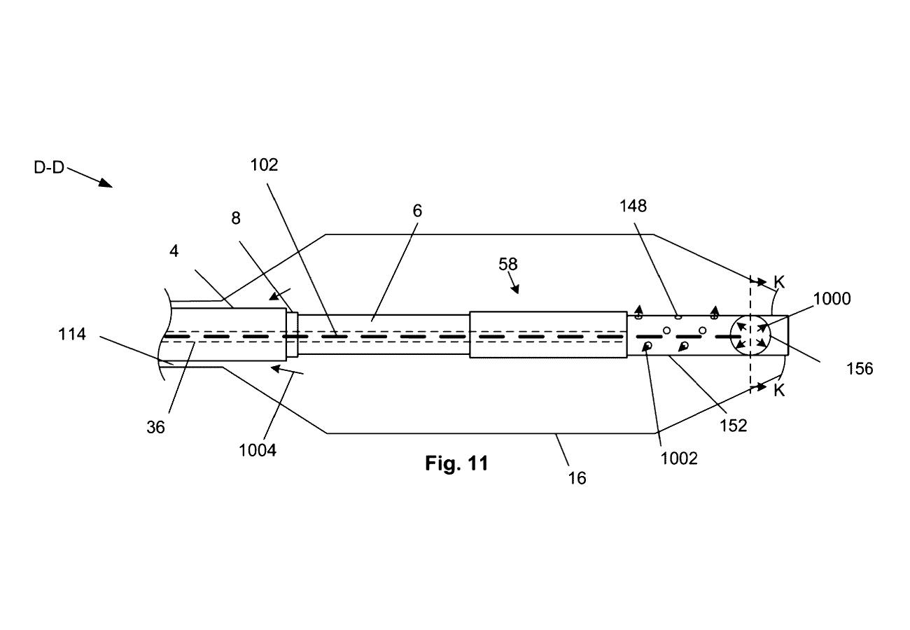

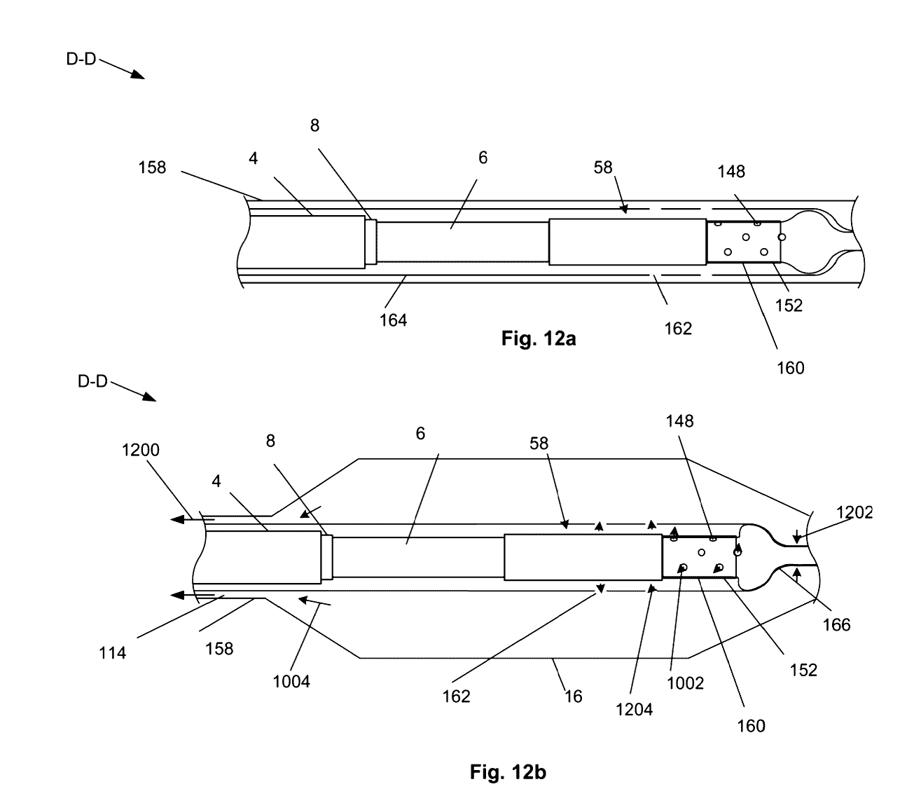

FIG. 12a illustrates that the apparatus 12 can have a rigid crimping outer tube 164 between the catheter 50 and cable jacket 4 and/or a rigid crimping catheter 158 with a cylindrical outer wall 110 and a crimping inner wall. The inner liner 152 can have fluid ports 148 and/or pores 176 (referred to throughout merely as fluid ports for explanatory purposes). The fluid pores 176 can be in porous ePTFE and can act like fluid ports 148, for example to allow fluid communication between the delivery and return flow channels 100 and the volumes radially exterior to the inner liner 152. The crimping outer tube 164 or crimping catheter 158 can have a crimping distal end with a tapering or narrowing radially inner surface or pinch wall 166. The pinch wall 166 can be distal to the fluid ports 148. The inner liner 152 and/or lubricious liner 42 can have a bulbous distal end more flexible than the tube and/or catheter 50. The inner liner 152 and/or lubricious liner 42 can have a reduced diameter distal to the fluid ports 148.

The outer tube 164 and/or crimping catheter 158 can have inflation ports 162 allowing fluid communication between the radial inside and radial outside environments of the tube and/or catheter 50, such as into and out of the external balloon 214.

The apparatus 12 can have a liner reinforcement 160 over, along, and/or within a length of the inner liner 152 extending distally from the antenna 58. The liner reinforcement 160 can be a collar or tube (bonded or not bonded to the inner liner 152), increased thickness (relative to the length distal to the reinforcement) of the inner liner 152, embedded or inter-weaved fiber reinforcements in the inner liner 152 (e.g., carbon fiber, steel fiber, Nitinol fiber), or combinations thereof.

FIG. 12b illustrates that the crimping tube and/or crimping catheter 158 can be proximally translated, as shown by arrows 1200, with respect to the inner liner 152 and/or lubricious liner 42. The pinch wall 166, outer tube 164, or crimping catheter 158 can then press against the outer surface of the inner liner 152 distal to the fluid ports 148, squeezing, compressing and closing, as shown by arrows 1202, the inner liner 152 and/or lubricious liner 42 distal to the fluid ports 148. Fluid flow in the inner fluid passageway can then exit the fluid ports 148 into the volume between the tube and the cable 2, as shown by delivery flow arrows. The fluid can then flow through the inflation ports 162 and into the balloon reservoir 56, as shown by arrows 1204, for example inflating the balloon 16. The fluid can then return flow 178, as shown by arrows 1004, out of the balloon 16, and between the tube and the catheter 50.

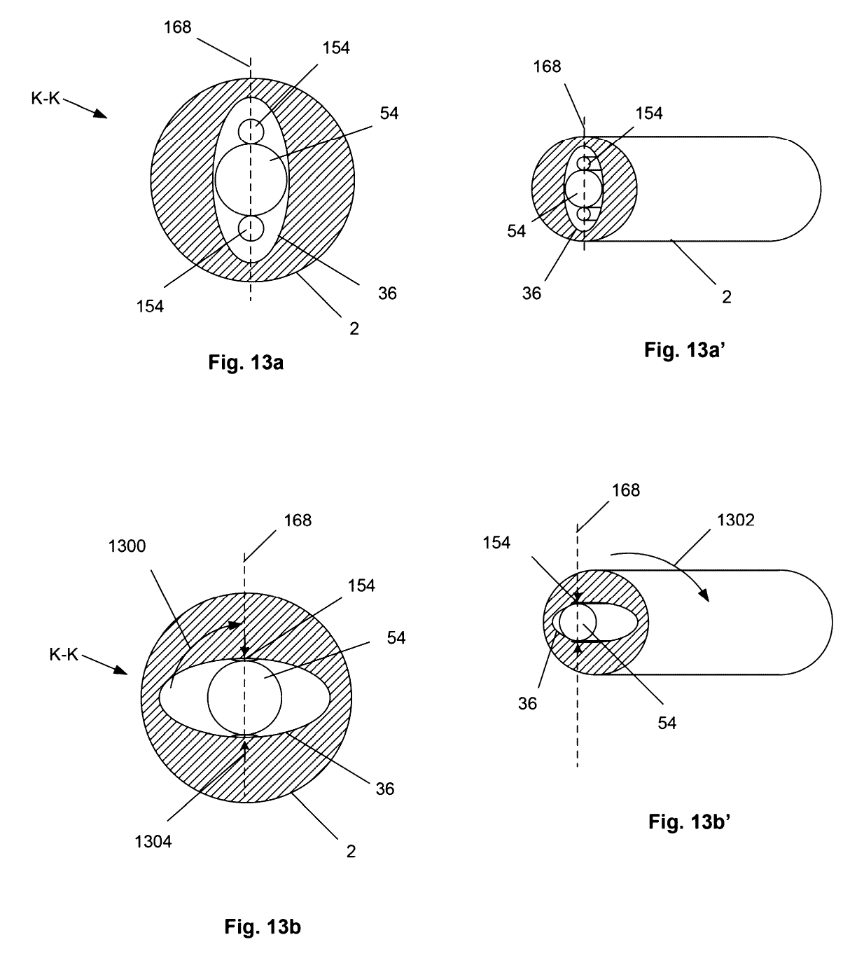

FIGS. 13a and 13a′ illustrates that the inner lumen 36 can have an oval keyhole cross-section. The fluid ports 148 can be proximal to cross-section K-K. The guidewire passageway 54 can be adjacent to one, two or more delivery flow passageways 154. For example first and second delivery flow passageways can be on diametrically opposite sides of the guidewire passageway 54. The passageways can each be surrounded by a respective liner. The guidewire passageway 54 liner can be less flexible or more rigid than the flow passageway liners. The combined passageways can have a long cross-sectional axis 168. When the open distal ports 174 of the flow passageways are in open configurations allowing flow out of the distal ports, the remainder of the cable 2, or the inner lumen 36 can otherwise be rotationally oriented with respect to the guidewire and flow passageways so that the long axis of the inner lumen 36 is aligned with the long cross-sectional of the combined passageways.

FIG. 13b illustrates that the inner lumen 36 can be rotated with respect to the guidewire and delivery flow passageways 154, as shown by arrow 1300. For example, as shown by arrow in FIG. 13b, the remainder of the cable 2 can be helically moved (i.e., rotated while being translated proximally), as shown by arrow 1302 (i.e., inclusive of the rotational the motion shown by arrow 1300), compared to the guidewire and delivery flow passageways 154. The long axis of the inner lumen 36 can be perpendicular to the combined passageway long axis. The flexible liners of the delivery flow passageways 154 can then be compressed or crimped partially or completely closed, as shown by arrows 1304. Fluid delivered through the delivery flow passageways 154 can then flow through the fluid ports 148 proximal to the crimp location and into the balloon reservoir 56, for example at lateral holes or ports, inflating the balloon 16.

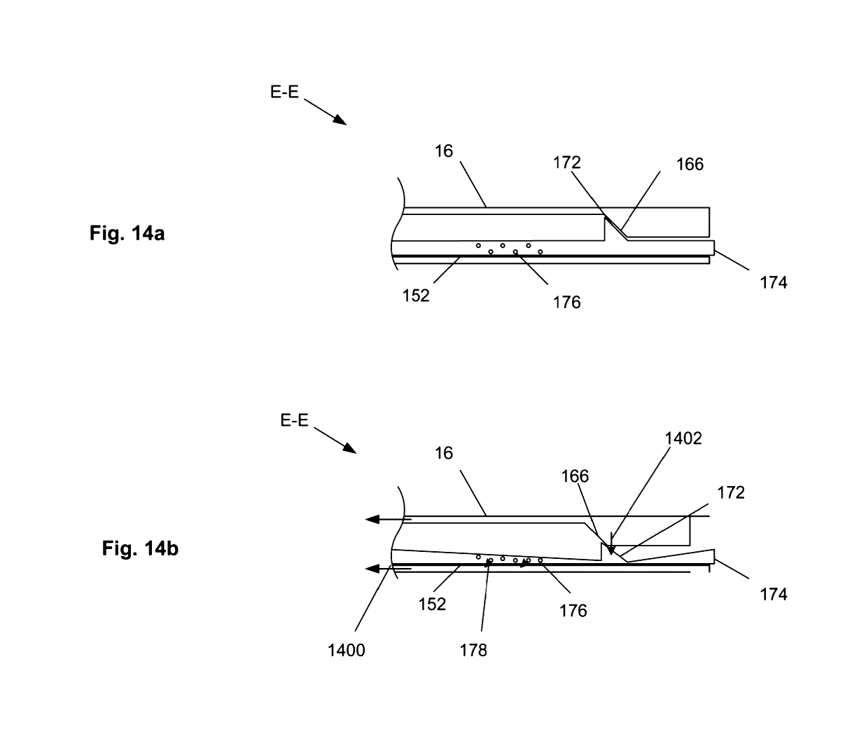

FIG. 14a illustrates that the inner liner 152, such as the lubricious liner 42, can have a crimp ramp 172 distal to an antenna 58 or the entire emitter 92. The crimp ramp 172 can extend radially outward from the surrounding inner liner 152. The crimp ramp 172 be unilateral as shown), angularly symmetric, or bilateral. The crimp ramp 172 can have a flat (as shown) or curved distal surface.

The inner liner 152 can have an open distal port 174.

The inner wall of the catheter 50 can have the pinch wall 166 positioned distal and adjacent to the crimp ramp 172. During use, a guidewire 102, tool, and/or fluids can be delivered through a fluid passageway and/or guidewire passageway 54 in the inner liner 152 and out the open distal port 174.

FIG. 14b illustrates that the catheter 50 can be retracted with respect to the inner liner 152, as shown by arrows 1400. The inner liner 152 can be more flexible or less rigid than the catheter 50. During, retraction, the crimp ramp 172 can slide against the pinch wall 166. The crimp ramp 172 can radially compress the liner, as shown by arrow 1402, for example occluding the delivery flow passageway 154 and forcing fluid flow through the inner fluid passageway out of the fluid ports 148, for example inflating the balloon 16 (not shown).

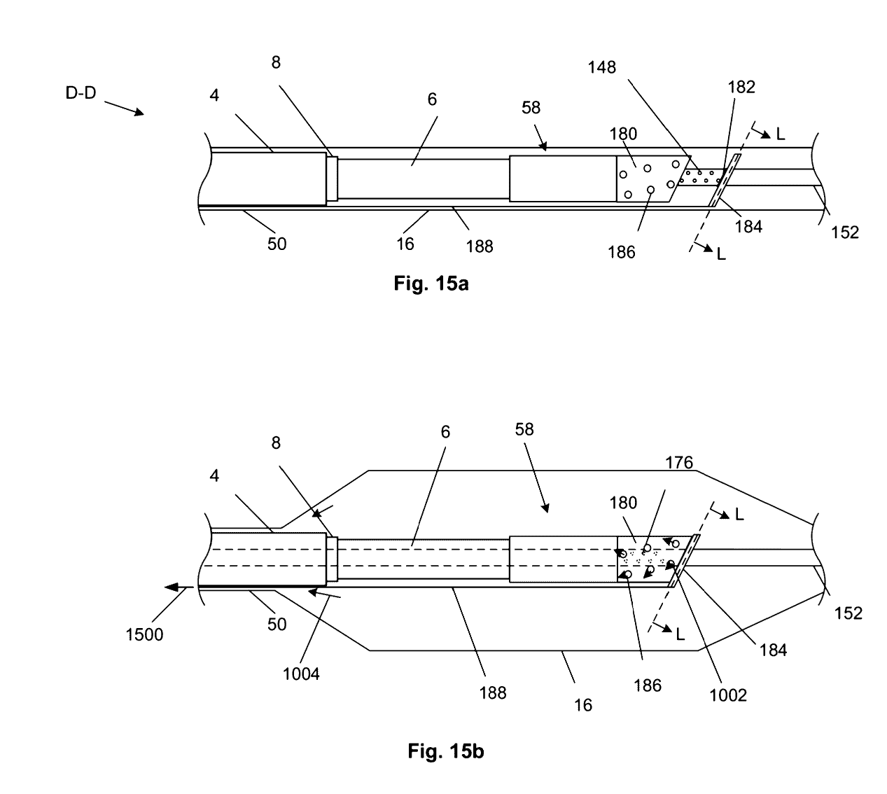

FIG. 15a illustrates a valve 184 can extend radially from the inner liner 152 distal to the fluid ports 148. The valve 184 can have a valve plane at a perpendicular or non-perpendicular (as shown) angle with respect to the longitudinal axis of the cable 2. The inner liner 152 can have a valve ridge 182. The valve ridge 182 can attach to the valve 184, for example fixing the valve 184 to the inner liner 152.

The apparatus 12 can have a spacer 180 attached to the distal end of the distal-most antenna 58. The spacer 180 can be an insulator. The spacer 180 can have lateral spacer ports 186 extending radially through the wall of the spacer 180.

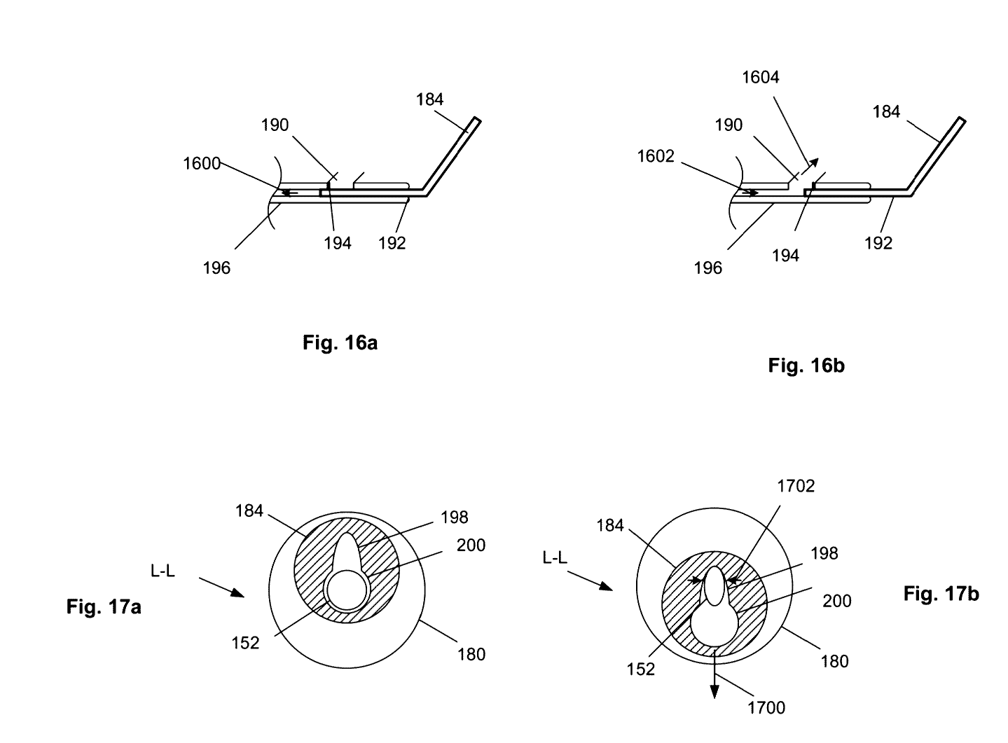

The valve 184 can be attached to a valve activation cord 188. The valve activation cord 188 can deliver a force to translate the valve 184. The valve 184 can be translated by fluid pressure, as shown in FIGS. 16a and 16b.

FIG. 15b illustrates that the valve activation cord 188 can be proximally translated, as shown by arrow 1500, to pull the valve 184 into a position to close distal passageway of the inner lumen 36. The valve 184 can close the delivery flow passageway 154, for example by translating down with respect to the liner, as shown in FIGS. 17a and 17b. Fluid delivered in the delivery flow passageways 154 in the inner liner 152 can then flow out of the fluid ports 148 into the spacer 180. The fluid can then flow out of the lateral spacer ports 186 and into the balloon 16, for example, inflating, the balloon 16.

FIGS. 16a illustrates that valve 184 can extend at an angle from a rigid control arm 192. The control area 192 can be inserted within a hydraulic valve activation track 196 fixed to the cable 2 and slidable within the track. The track can have a track outlet 190 exiting the track perpendicular or other non-zero angle to the longitudinal axis of the control arm 192. The control arm 192 can have a valve stop 194 extending perpendicularly and fixed to the remainder of the control arm 192. The valve stop 194 can extend into the track outlet 190.

When the track is exposed to fluid suction, the suction pressure can pull and translate the control arm 192 proximally, as shown by arrow 1600, until the valve stop 194 interference fits against the proximal side of the track outlet 190. The control arm 192 can block or cut off fluid communication between the track outlet 190 and the track, sealing the track from the track outlet 190. The valve 184 can then be in a closed configuration, for example, pulled against the spacer 180.

FIG. 16b illustrates that when positive fluid pressure is delivered to the track, the fluid can press and translate the control arm 192 distally, as shown by arrow 1602, until the valve stop 194 interference its against the distal side of the track outlet 190. The track. outlet 190 (i.e., valve) can then be open and in fluid communication with the track and fluid can be delivered through the track and out the track outlet 190, as shown by arrow 1604.

The track outlet 190 can flow directly or indirectly into the balloon 16. For example, the track outlet 190 can flow into the delivery flow passageway 154 in the inner liner 152 or can flow directly into the balloon reservoir 56.

FIG. 17a illustrates that the valve 184 can have a keyhole with a keyhole crimp 198 and a keyhole slot 200. The keyhole slot 200 can have a diameter equal to or greater than the inner liner 152 and/or lubricious liner 42. The keyhole crimp 198 can have a tapering, narrowing width, narrower than the diameter of the keyhole slot 200. When the valve 184 is in the open configuration, the inner liner 152 can extend through the keyhole slot 200 and be patent and un-crimped.

FIG. 17b illustrates that the valve 184 can translate down compared to the inner liner 152, as shown by arrow 1700. For example, when the valve 184 is pulled proximally compared to the liner, the angle of the valve 184 can increase with respect to the longitudinal axis of the cable 2. As described herein, the valve 184 can be actuated from a direct mechanical linkage and/or a hydraulic system (i.e., fluid pressure). The liner can then be forced from the keyhole slot 200 into the keyhole crimp 198. The keyhole crimp 198 can then crimp or compress, as shown by arrow 1702, the inner liner 152. The liner and distal fluid delivery passageway can be crimped or compressed partly or completely closed, for example, routing fluid flow into the balloon 16.

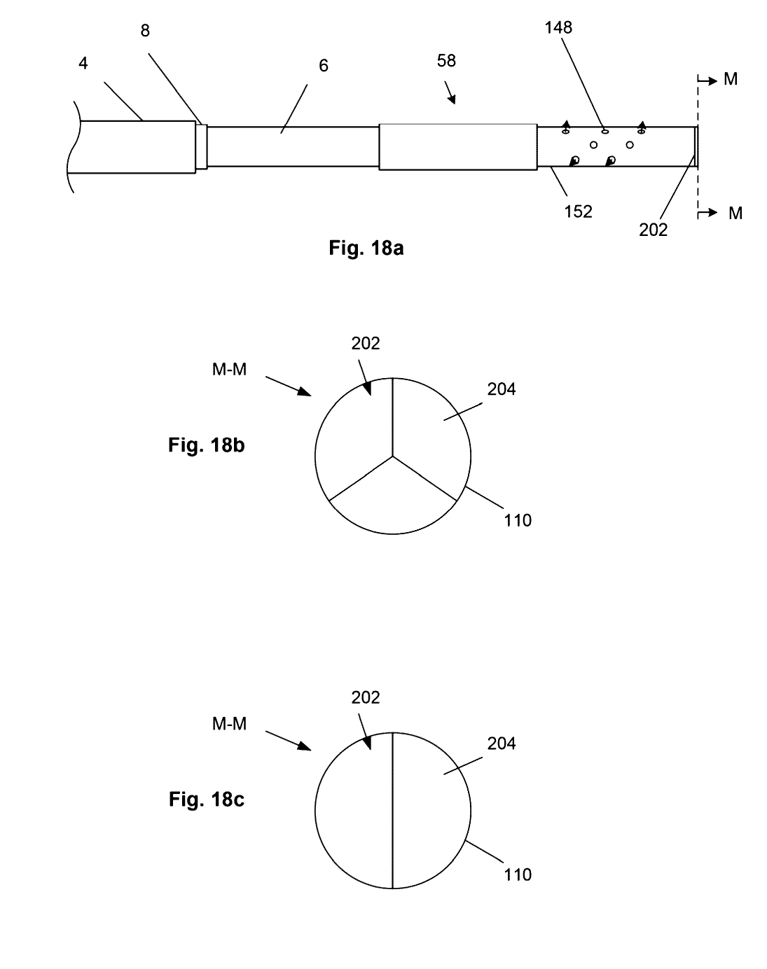

FIG. 18a illustrates that the open distal port 174 of the inner liner 152 can be covered by a distal cap valve 202. The distal cap valve 202 can be attached to the distal terminal face of the inner liner 152. The distal cap valve 202 can have a diameter equal to or greater than the inner liner 152. The distal cap valve 202 can remain closed due to fluid pressure in the flow passageway of the inner liner 152, and can be opened from the insertion force of the guidewire 102. When closed the distal cap valve 202 can route fluid flow through the fluid ports 148 and to the balloon 16, as shown by arrows.

FIG. 18b illustrates that the distal cap valve 202 can have a tricuspid configuration having three evenly angularly distributed, leaflets 204, each forming a 120° angle from the center.

FIG. 18c illustrates that the distal cap valve 202 can have a duckbill, bicuspid, or mitral configuration having two evenly angularly distributed leaflets 204, each forming a 180° angle from the center.

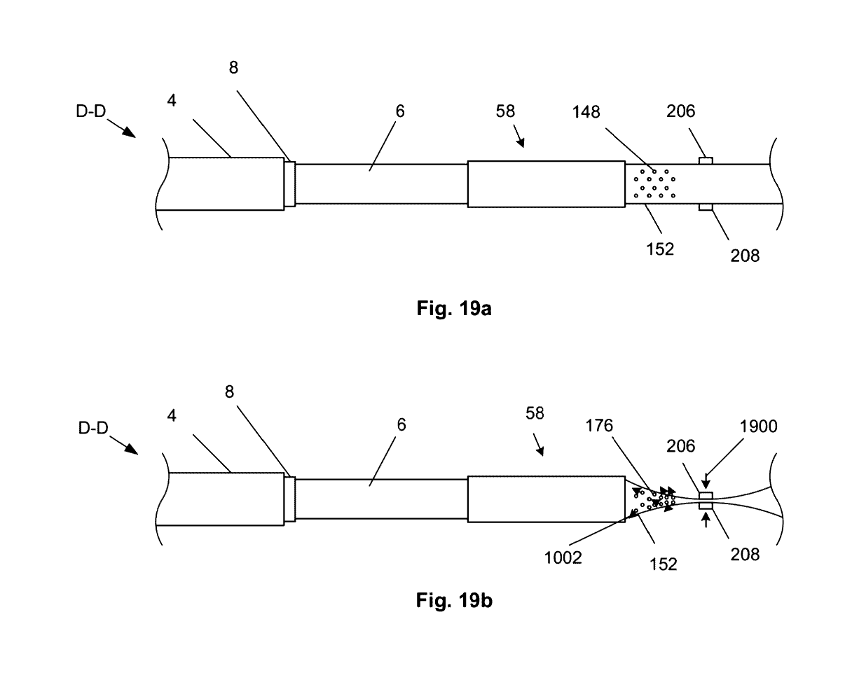

FIG. 19a illustrates that the distal end of the inner liner 152 can have a first magnet 206 and a second magnet 208 distal to the fluid ports 148. The magnets can be on the radial inside, radial outside, or embedded in the liner wall. The first magnet 206 can be diametrically opposite to the second magnet 208. The magnets can be electro-magnets and/or permanent magnets. When the liner is in an open or patent configuration, the magnets can be inactive or restrained configuration.

FIG. 19b illustrates that the first 206 and second 208 magnets can be inductively activated by an inductive power source. The inductive power source can be located inside or outside of the patient's body. The first magnet 206 and second magnet 208 can be drawn together by magnetic force, as shown by arrows 1900. The first magnet 206 and second magnet 208 can crimp or compress the inner liner 152 distal to the fluid ports 148, blocking or obstructing fluid flow out of the open distal port 174 and through the fluid ports 148 to the balloon 16, as shown by arrows.

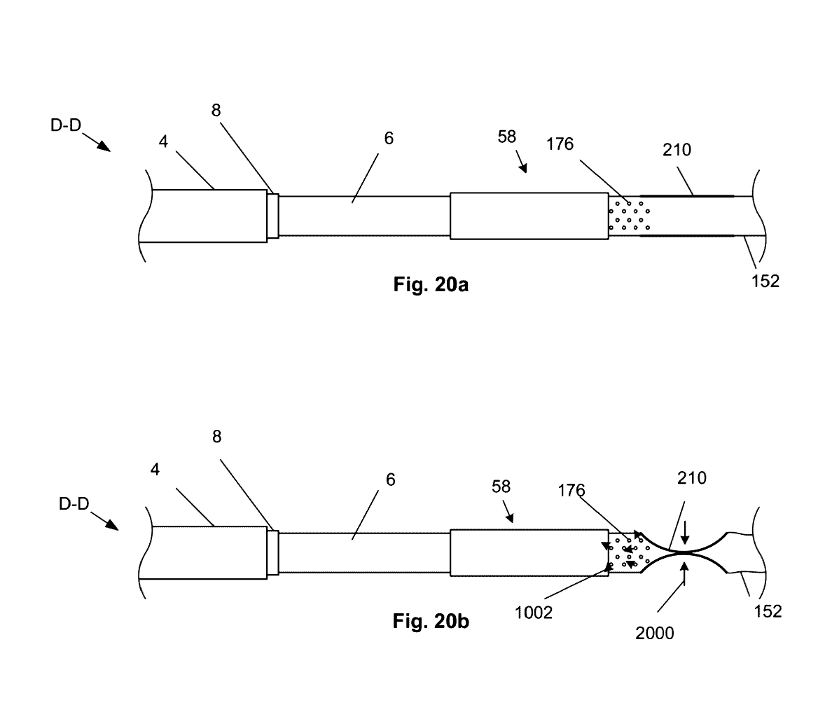

FIG. 20a illustrates that the distal end of the inner liner 152 can have one or more (shown with two diametrically opposed) shape memory springs 210 For example, the shape memory springs 210 can be made from a nickel titanium alloy (e.g., Nitinol). The shape memory springs 210 can be on the radial inside, radial outside, or embedded in the liner wall. The shape memory springs 210 can be in straight configurations when the liner is in an open or patent configuration. The guidewire 102 can be inserted in the inner liner 152 to deform the shape memory springs 210 into the straight configurations.

FIG. 20b illustrates that the shape memory springs 210 can be biased to curl and collapse or deform toward the radial center of the liner, as shown by arrows, for example when guidewire 102 is removed from the guidewire passageway 54 in the inner lumen 36 in the liner. The shape memory springs 210 can squeeze or crimp the inner liner 152 completely or partially closed, blocking or obstructing fluid flow out of the open distal port 174 and through the fluid ports 148 to the balloon 16, as shown by arrows 2000.

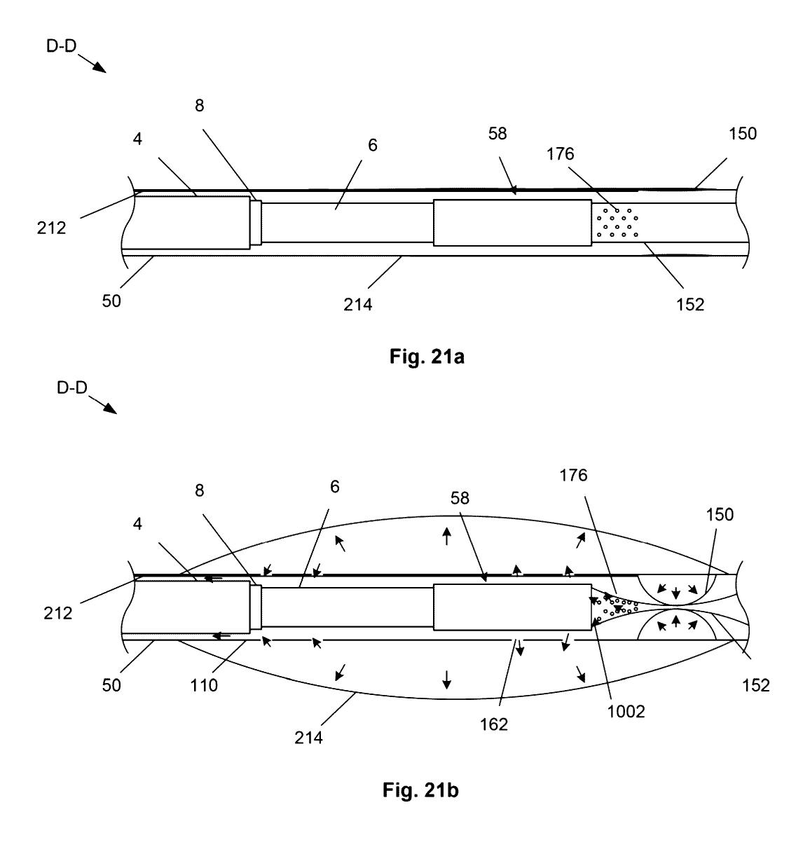

FIG. 21a illustrates that the catheter 50 can have one or more crimping balloon or bladder inflation channels 146 extending from a proximal pressurized fluid source. For example, the apparatus 12 can have a single inflation channel 212 can have a tube shape and circumscribe or encircle the cable 2 and emitter 92, the apparatus 12 can have more than one inflation channel 212, with each channel symmetrically arranged around the cable longitudinal axis. The inflation channels 212 can extend from and be attached to the catheter 50.

The catheter outer wall 110 and/or the distal ends of the inflation channels 212 can be attached to one or more crimping, inflatable internal balloons or bladders distal to the fluid ports 148 and extending radially inward. The inflatable bladders 150 can be in fluid communication with the inflation channels 212. When the inflatable bladders 150 are in deflated configurations, the flow passageways in the inner liner 152 can be open and patent, allowing fluid to flow to the open distal port 174. The inflatable bladders 150 can be, for example, bilaterally positioned on diametrically opposite sides of the inner liner 152, or toroid-shaped encircling the inner liner 152. The toroid-shaped inflatable bladders 150 can have flat radial exteriors when in an inflated configuration.

The catheter outer wall 110 and/or the radially outer surface of the inflation channels 212 can be attached to one or more external balloons 214 longitudinally extending proximally from the cable 2 to distal to the emitter 92. The inflation channels 212 can be in direct or indirect fluid communication with the external balloons 214.

The outer walls 110 can have inflation ports 162, as shown and described, in FIGS. 12a and 12b.

FIG. 21b illustrates that the inflatable bladders 150 can be inflated by fluid delivered through the inflation channel 212, as shown by arrows. The inflatable bladders 150 can crimp, pinch, compress and partially or completely close the flow passageways in the inner liner 152, forcing fluid in the delivery flow passageways 154 to flow through the fluid ports 148, as shown by arrows. The fluid can then flow through the inflation ports 162, inflating the external balloon 214, as shown by arrows. The fluid in the balloons 16 can flow through inflation ports 162 and through one or more return flow passageways 114 between the cable jacket 4 and the catheter 50. The inflated external balloons 214 can space the emitter 92 equidistantly from surrounding lumen walls, centering the emitter 92 in a target lumen.

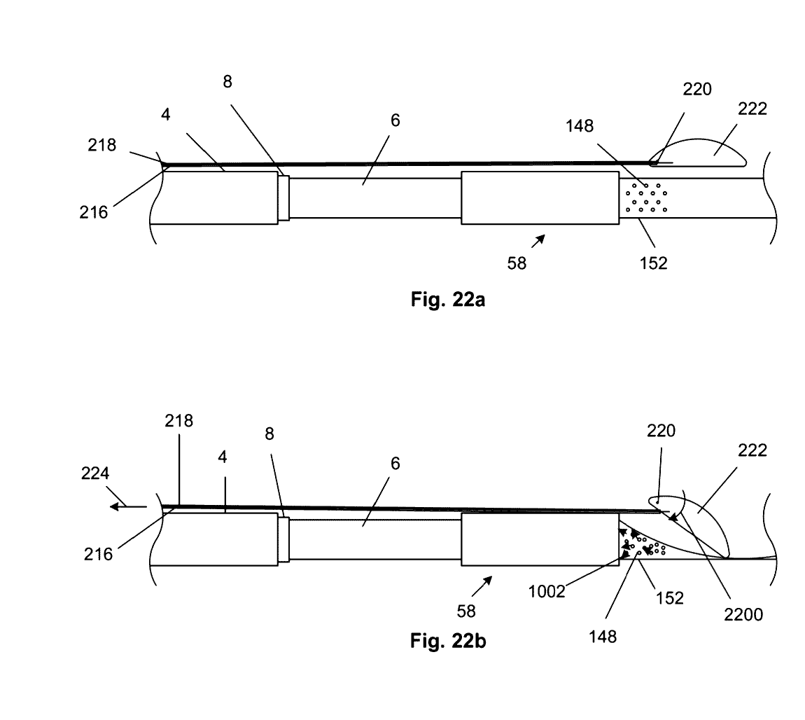

FIG. 22a illustrates that the apparatus 12 can have a cord tube 216 extending parallel to the cable 2 from a control interface at the proximal end of the apparatus 12 or merely extending freely out of a port at the proximal end of the apparatus 12 to the distal end of the device inside of or distal to the balloon 16. The cord tube 216 can be fixed to the cable 2. The apparatus 12 can have a cam activation cord 218 longitudinally slidable in the cord tube 216. The apparatus 12 can have a crimping cam 222 rotatably attached to the cam activation cord 218. The crimping cam 222 can have a cam axle 220 rotatably attached to the catheter 50 and/or the cord tube 216. The cam axle 220 can be transverse to the cable longitudinal axis. The end of the crimping cam 222 farther away from the cam axle 220 can be distal to the fluid ports 148. The distal terminal end of the cam activation cord 218 can be attached to the cam at a torque-arm distance away from the cam axle 220.

FIG. 22b illustrates that the cam activation cord 218 can be pulled and. translated proximally 224 relative to the cord tube 216, as shown by arrow. The cam activation cord 218 can impart a torque on the crimping cam 222, rotating the crimping cam 222, as shown by arrow 2200. The crimping cam 222 can press, compress, crimp, and pinch the inner liner 152. The crimping, cam 222 can crimp, pinch, compress and partially or completely close the flow passageways in the inner liner 152, forcing fluid in the delivery flow passageways 154 to flow through the fluid ports 148, as shown by arrows. The fluid can then flow through the inflation ports 162, inflating the external balloon 214, as shown by arrows 1002.

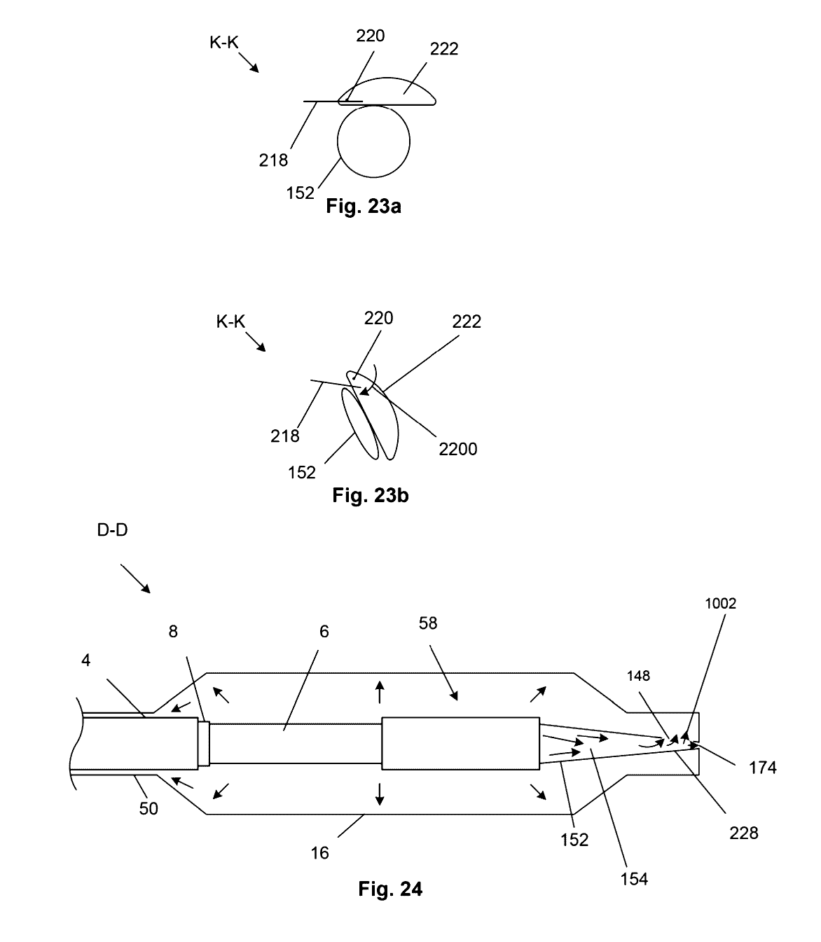

FIG. 23 illustrates that the crimping cam 222 can be oriented transverse to the cable longitudinal axis (compared with the orientation of the crimping cam 222 of FIGS. 22a and 22b parallel with the cable longitudinal axis). The cam axle 220 can be transverse to the cable longitudinal axis. The crimping cam 222 can be rotated as shown by arrow 2200, for example, squeezing closed the inner liner 152 across the transverse cross-section of the inner liner 152.

FIG. 24 illustrates that the delivery flow passageway 154 distal to the distal-most antenna 58 can radially narrow or taper 228 relative to the distal length of the delivery flow passageway 154. The delivery flow passageway can be narrower at the open distal port 174 than at the distal terminal end of the distal-most antenna 58. The distal port can be completely closed. The inner liner 152 distal to the antenna 58 can be elastic or attached to an elastic band that can radially constrict the inner liner 152. The flow resistance can force the majority of fluid delivered through the delivery flow passageway 154 to flow through the lateral fluid port and into the balloon 16, as shown by arrow 1002.

FIG. 25a illustrates that a choke cord 230 can extend parallel to the cable 2 from a control interface at the proximal end of the apparatus 12 to the distal end of the device inside of or distal to the balloon 16. The choke cord 230 can be in a cord tube 216 as described above for FIGS. 22a and 22b.

The inner liner 152 can have a cord channel 234 distal to the inner liner 152. The cord channel 234 can be open at a proximal end of the cord channel 234 at a cord channel entry port 232. The distal end of the choke cord .230 can be in the cord channel 234 and can extend from the cord channel 234 at the cord channel entry port 232. The cord channel 234 can helically wind, loop, or rotate around the inner liner 152 distal to the fluid ports 148. The choke cord 230 can helically wind, loop, or rotate around the inner liner 152 distal to the fluid ports 148 inside of the cord channel 234 or, for variations without a cord channel 234. the outer surface of the inner liner 152.

The distal terminal end of the choke cord 230 can be fixed to the inner liner 152 at a cord fixation point 236. The cord fixation point 236 can be inside the cord channel 234 (e.g., at the distal terminal end of the cord channel 234) or distally beyond the termination of the cord fixation channel.

FIG. 25b illustrates that the choke cord 230 can be translated proximally, as shown by arrow 238. When the choke cord 230 is translated proximally, the windings of the choke cord 230 can cinch the inner liner 152 distal to the fluid ports 148 causing the inner liner 152 to contract, as shown by arrows 2500. The flow resistance due to the radially contracted inner liner 152 can force the majority of fluid delivered through the delivery flow passageway 154 to flow through the lateral fluid port and into the balloon 16.

The choke cord 230 and/or the length of the inner liner 152 collinear with the choke cord 230 winds or loops can be made all or partially from a resilient material (e.g., Nitinol). When proximal force is released from the choke cord 230, the inner liner 152 distal to the fluid ports 148 can radially expand to a pre-contracted configuration.

Cords described herein can be flexible monofilament or multifilament (e.g. braid) leaders (e.g., woven PTFE filaments), single-link or multi-link rods, or combinations thereof.

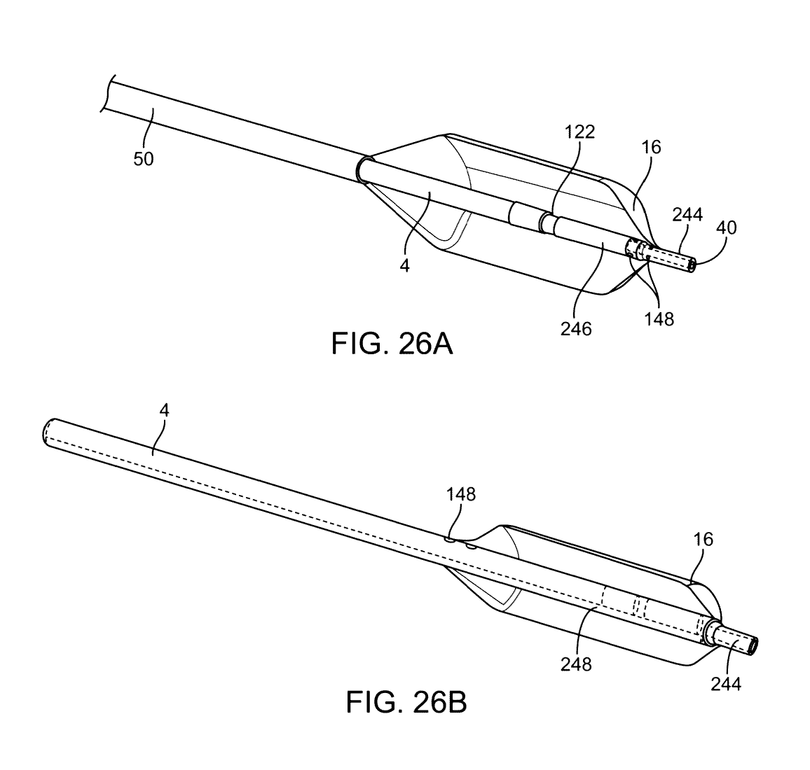

FIG. 26a illustrates that the distal terminal end of the apparatus 12 can be an apparatus distal tip 244. The distal end of the balloon 16 can attach to the apparatus distal tip 244. The apparatus distal tip 244 can be the distal end of the inner liner 152.

The distal terminal end of the guidewire passageway 54 can have a guidewire port 40 through radial center of the cable 2 at the apparatus distal tip 244. During use, the guidewire 102 can extend through the radial center of the cable 2 and distally exit at the radially center of the cable 2.

FIG. 26b illustrates that the guidewire passageway 54 can be in a guidewire tube 248 attached to the lateral side of the cable 2. The guidewire tube 248 can be radially outside of the cable 2. The guidewire tube 248 longitudinal axis can be parallel and off-center from the cable longitudinal axis.

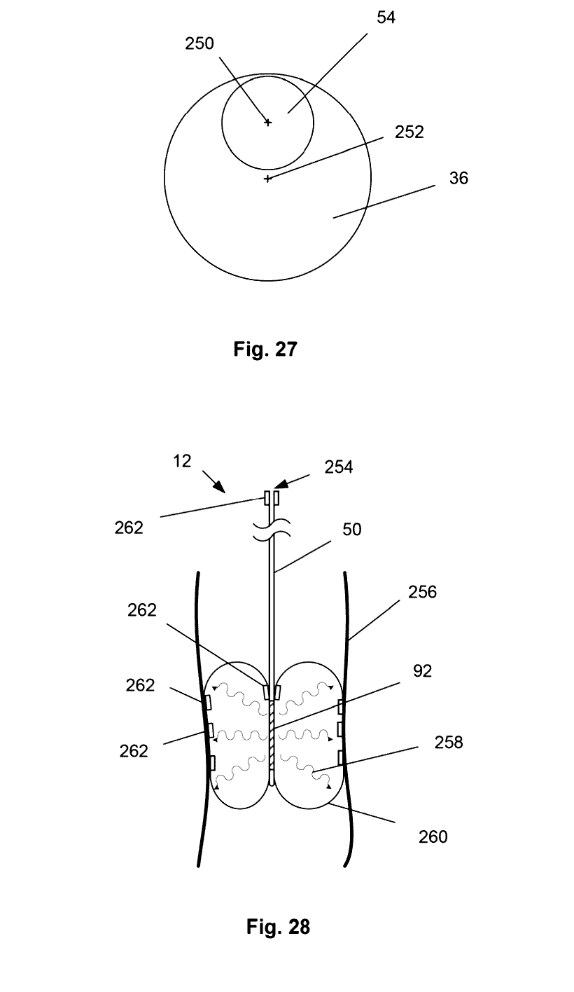

FIG. 27 illustrates that guidewire passageway center 250 can be offset from the inner lumen center 252 (e.g., the cable longitudinal axis), and radially inside or outside of the cable 2. The center of the balloon 16 can then be offset for unilateral energy delivery, for example for unilateral Barret's Esophagus.

FIG. 28 illustrates that the apparatus 12 can have temperature sensors (e.g., thermocouples 262, thermistors, optical thermocouples, or combinations thereof) on the inside and/or outside and/or embedded into the wall of the balloon 16. The balloon 16 can be inflated within a biological vessel. The temperature sensors can be centrally located on the inflatable balloon 260 in contact with the vessel wall 256. The temperature sensors can be angularly and longitudinally symmetrically located on the balloon 16. The temperature sensors can be on the proximal-most portion of the balloon 16 attached to the catheter 50.

The apparatus 12 can have a fluid input port 254 where fluid is delivered into fluid passageways. The apparatus 12 can have temperature sensors on the inside and/or outside of the fluid input port 254.

The temperature sensors can sense the temperature at the respective location and communicate the temperature over a wired or wireless connection to a processing unit, for example for analysis and/or display to the user of the apparatus 12. The processing unit can increase the fluid flow rate through the fluid passageways and/or decrease the delivered fluid temperature if the sensed temperatures exceed a threshold maximum temperature. The processing unit can adjust the transmitted microwave 258 power. The processing unit can decrease the fluid flow rate through the fluid passageways and/or increase the delivered fluid temperature if the sensed temperatures fail to exceed a threshold minimum temperature.

The apparatus 12 can create target tissue temperatures from about 37° C. to about 100° C. for microwave energy exposure times from about 30 seconds to about 600 seconds. For example, the apparatus 12 can be configured to expose target tissue to microwave energy from about 30 seconds to about 150 seconds, resulting, in a target tissue temperature from about 50° C. to about 70° C. The fluid flow rate in the apparatus 12 can be from about 0 ml/min to about 100 ml/min. The fluid temperature in the balloon 16 can be from about 0° C. to about 37° C. more narrowly from about 0° C. to about 25° C., for example about 37° C.

The emitter 92 can transmit microware energy 258 through the balloon 16 and fluid in the balloon 16 to the vessel wall 256 and surrounding tissue. For example, the microwave energy 258 can be directed at the vessel wall 256 and/or a target tissue (e.g., a target nerve) on or under e the vessel wall 256. If the apparatus 12 is configured for the guidewire 102 to be inserted through an inner lumen 36, the guidewire 102 can be removed from the inner lumen 36 before the transmission of microwave energy 258 by the emitter 92.

The emitter 92 (e.g., antenna 58) can be used as a microwave receiver. The emitter 92 can receive or absorb the microwave energy radiated from the target tissue for radiometric sensing. The received energy can be measured by one or more radiometer circuits which translate the measured microwave thermal power into a voltage. The voltage can additionally be digitized by analog to digital converter circuits in a processing unit. The radiometric circuits can be housed together or separately from the analog to digital converter circuits.

While connected to the power source, the cable 2 can interface with a receiving circuit. The receiving circuit can use received (by the emitter 92) and/or measured (by the emitter 92) energy, at one or multiple frequencies, from the treatment sight to convert the received energy readings to temperature or energy measurements of treated area. The passageways can contain sensors or materials necessary for radiometry system calibration or offset.

The fluid ports 148 can be filled with porous material, such as porous ePTFE.

The inner liner 152 can be the lubricious liner 42 or the apparatus 12 can have separate inner liners 152 and lubricious liners 42.

International Application No. PCT/US2014/021233, filed 6 Mar. 2014, U.S. Provisional Application No. 61/775,281, filed Mar. 8, 2013, and U.S. patent application Ser. No. 14/199,374, filed Mar. 6, 2014, are all incorporated by reference in their entireties, and any variations and/or elements of the aforementioned applications can be used in combination with the variations and elements described, elsewhere herein, for example but not limited to balloons 16 that can inflate for fixation and perfusion balloon 16 variations (e.g., having one or more channels for blood or fluid flow to continue distal to the balloon 16 and into the biological lumen outside of the apparatus 12) and elements described in the aforementioned applications.

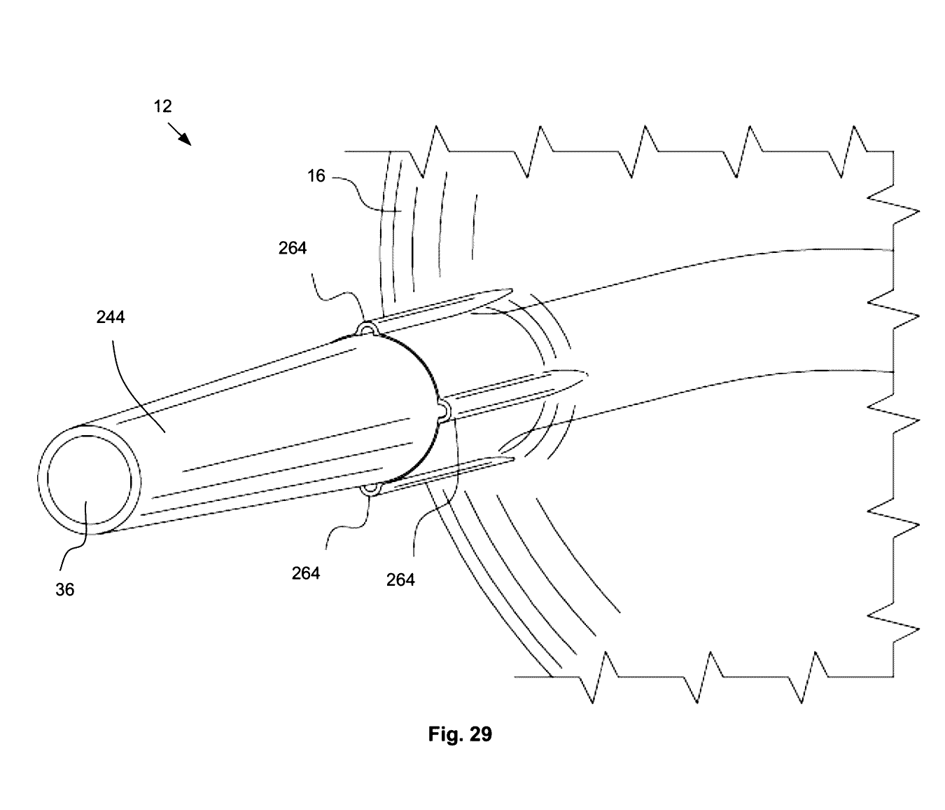

FIG. 29 illustrates that the apparatus 12 can have one or more (e.g., three or four, as shown) irrigation ports 264 at the junction between the inflatable balloon 260 and the apparatus distal tip 244. The irrigation ports 264 can be radially outside of the apparatus distal tip 244. Fluid in the balloon 16 can flow through the irrigation ports 264 and directly into the target lumen, such as an esophagus or blood vessel. For example, the apparatus 12 can directly perfuse the target location with fluid from the inside of the balloon 16. The irrigation ports 264 can be in direct communication with one or more cooling channels, for example delivering cooled saline solution through the irrigation ports 264 and into the target location.

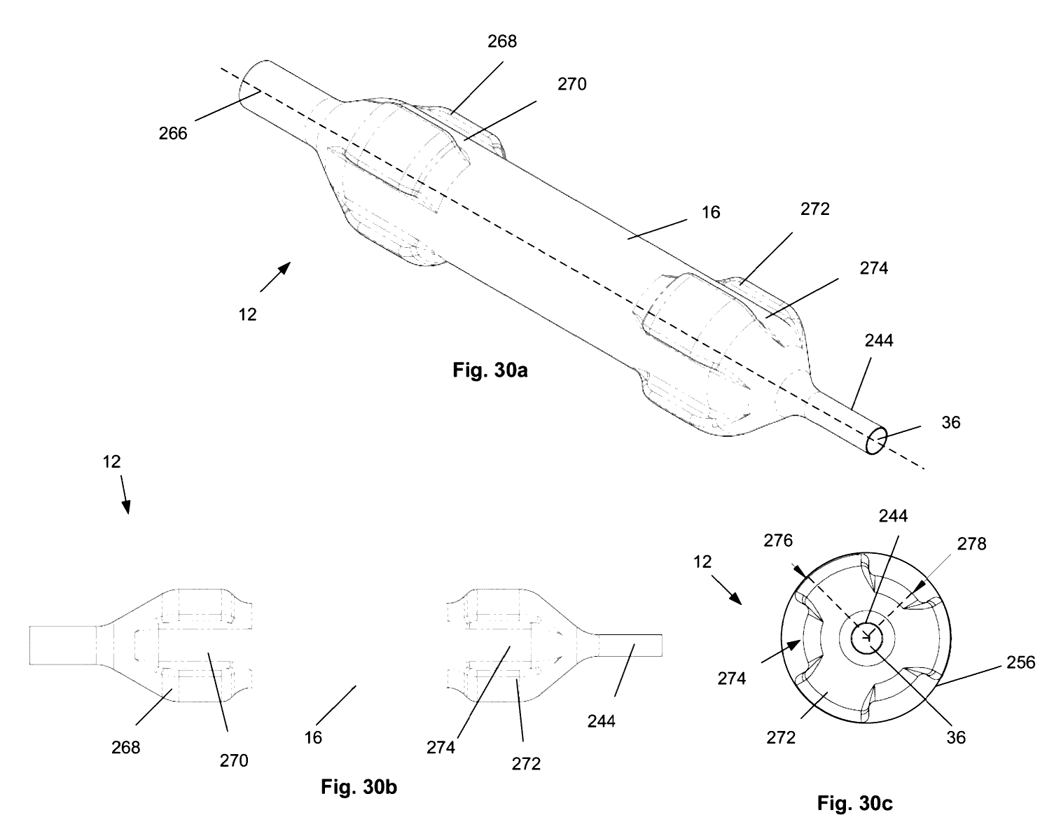

FIGS. 30a through 30c illustrate that the balloon 16 can have one or more (e.g., two or three lobes evenly angularly spaced around the balloon 16) balloon lobes 280 formed on the radially outer surface of the balloon 16 and extending radially outward from the remainder of the balloon 16. The lobes can extend longitudinally parallel with the balloon longitudinal axis 266. The balloon 16 can have lobes extending the entire length of the balloon 16 or part of the length of the balloon 16. For example, the balloon 16 can have one or more balloon proximal lobes 268 and one or more balloon distal lobes 272, as shown.

The balloon 16 can have one or more (e.g., two or three lobes evenly angularly spaced around the balloon 16) inter-lobe recesses 282 between angularly adjacent lobes. For example, the balloon 16 can have distal inter-lobe recesses 274 or cooling channels angularly between adjacent balloon distal lobes 272 and proximal inter-lobe recesses 270 angularly between adjacent balloon proximal lobes 268, as shown.

The balloon 16 can have angularly identical sets of proximal and distal lobes, for example three angularly evenly spaced distal lobes and three angularly evenly spaced proximal lobes where the proximal lobes are angularly aligned with the distal lobes. The balloon 16 can have angularly offset sets of proximal and distal lobes, for example three angularly evenly spaced distal lobes and two angularly evenly spaced proximal lobes; or three angularly evenly spaced distal lobes and three angularly evenly spaced proximal lobes wherein the proximal lobes are angularly offset from the distal lobes (e.g., the proximal inter-lobe recesses 270 can angularly align with the distal lobes).

FIG. 30c illustrates that an the balloon 16 can be inflated in a vessel so the lobes extend radially to the vessel wall 256. The vessel wall 256 can have an inner radius approximately equal to a lobe radius 276. The lobe radius 276 can be from about 1 mm to about 9 mm, for example about 3 mm. The radially outer surface of the lobe can contact, press, and seal against the radially inner surface of the vessel wall 256. The inter-lobe recess 282 can have an inter-lobe recess radius 278 that can be smaller than the vessel wall 256 inner radius. The inter-lobe recess radius 278 can be from about 0.5 mm to about 8 mm example about 2.5 mm. The inter-lobe recesses 282 can act as flow-through channels for fluids flowing through the biological vessels, allowing the fluids to longitudinally flow past the balloon 16 (i.e., from proximal of the balloon 16 to distal to the balloon 16) when the balloon 16 is in an inflated configuration in the vessel.

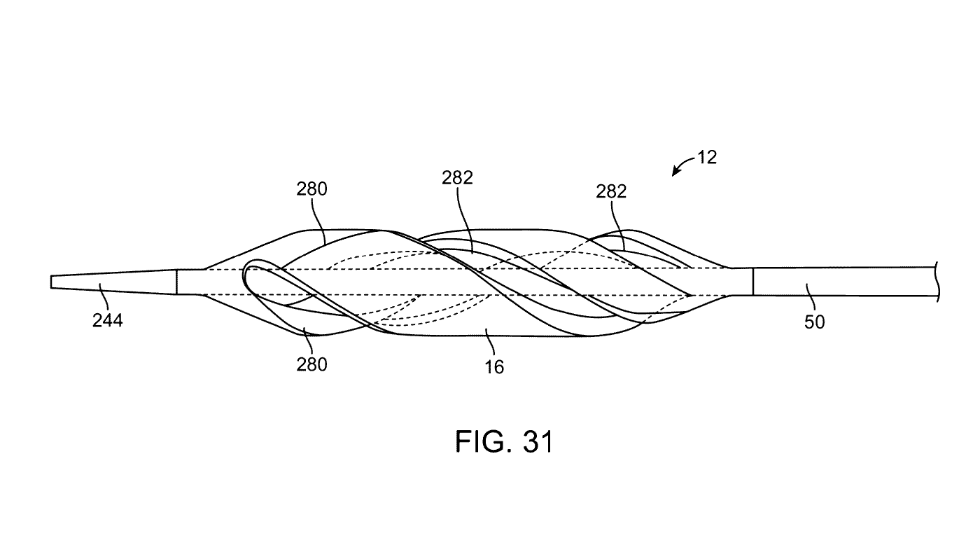

FIG. 31 illustrates that balloon 16 can have one or more balloon lobes 280 extending helically along the balloon 16. The one or more inter-lobe recesses 282 can extend helically along the balloon 16.

The balloon 16 can have a burst or failure pressure of for example, about 12 atm.

Any elements described herein as singular can be pluralized (i.e., anything described as “one” can be more than one), Any species element of a genus element can have the characteristics or elements of any other species element of that genus. The above-described configurations, elements or complete assemblies and methods and their elements for carrying out the invention, and variations of aspects of the invention can be combined and modified with each other in any combination.

We claim:

1. A method for delivering microwave energy to a target biological tissue comprising:
positioning a guidewire adjacent to the target biological tissue;
delivering a coaxial cable comprising a microwave emitter over the guidewire, wherein the cable has a longitudinal axis, and wherein the cable comprises an inner conductor, an outer conductor insulated from and radially outside of the inner conductor, and a lumen radially inside of the outer conductor, and wherein the lumen extends through the emitter, and wherein the guidewire slides through the lumen;
removing the guidewire from the lumen; and
delivering a fluid to the lumen, wherein the fluid flows in the first lumen longitudinally distal to the emitter.

2. The method of claim 1, further comprising after the fluid flowing in the first lumen, the fluid then flowing radially outside of the emitter, and then flowing in a fluid passageway proximal to the emitter.

3. The method of claim 1, wherein the delivering of the fluid occurs after the removing of the guidewire.

4. A method for delivering microwave energy to a target biological tissue comprising:
delivering a coaxial cable adjacent to the target biological tissue, wherein the cable has a longitudinal axis and comprises a microwave emitter, an inner conductor, an outer conductor insulated from and radially outside of the inner conductor, and a first passageway extending through the coaxial cable, wherein the first passageway has a port distal to the emitter;
delivering a fluid through the first passageway and the port; and
transferring a microwave energy from the antenna to the target biological tissue, and wherein the delivering the fluid occurs concurrently with the transferring of the energy.

5. The method of claim 4, further comprising occluding the end of the first passageway distal to the emitter, wherein the occluding comprises dosing fluid-tight.

6. The method of claim 4, wherein a balloon is in fluid communication with the first passageway.

7. The method of claim 6, further comprising inflating the balloon, wherein the inflating further comprising selectively positioning the antenna in a biological vessel adjacent to the target biological tissue.

8. The method of claim 4, further comprising detecting a temperature of biological tissue at or adjacent to at least one of the target biological tissue, fluid, emitter, coaxial cable, a power input connector, or a magnetic field, emitted by the emitter, or thermal radiation by the biological tissue measured by the emitter.

9. The method of claim 8, wherein the delivering of the fluid further comprises delivering the fluid at a flow rate, and controlling the flow rate based at least in part on the detected temperature of at least one of the biological tissue, fluid, emitter, coaxial cable, a power input connector, or a magnetic field emitted. by the emitter, or thermal radiation by the biological tissue measured by the emitter.

10. The method of claim 6, further comprising inflating a. balloon outside of the antenna.

11. The method of claim 4, wherein the coaxial cable comprises a dielectric between the inner conductor and the outer conductor, and wherein the first passageway extends through the dielectric.

12. The method of claim. 4, wherein the coaxial cable comprises a second passageway, and further comprising delivering the fluid to the second passageway.

13. The method of claim 4, further comprising inserting a guidewire, introducing the fluid, and connecting a power source to a connector at a proximal terminal end of the coaxial cable.

14. The method of claim 13, wherein the connector comprises an impedance matching circuit extending from a power input extension in the connector.

15. A method for delivering microwave energy to a target biological tissue comprising:
delivering a catheter adjacent to the target biological tissue, wherein the catheter comprises a balloon at a distal end of the catheter, and wherein the delivering comprises positioning the balloon adjacent to the target biological tissue;
delivering a coaxial cable adjacent to the target biological tissue, wherein the coaxial cable is inside of the catheter, and wherein the cable comprises a microwave emitter, and wherein the emitter is positioning adjacent to the target biological tissue, and wherein the emitter has an emitter longitudinal axis, and wherein, the coaxial cable has a first passageway radially inside the emitter;
further comprising actively circulating, fluid through the passageway, distal to the emitter, radially outside of and longitudinally coincidental with the emitter, and through the catheter proximal to the emitter and the balloon, wherein the fluid can be delivered at least one of toward the distal end of the passageway, toward the proximal end of the passageway, or in alternating directions.

16. The method of claim 15, wherein circulating the fluid through the catheter comprises flowing the fluid through a second passageway defined between the radial outside of the cable and the radial inside of the catheter.

17. The method of claim 15, wherein circulating the fluid through the catheter comprises flowing the fluid through a second passageway radially inside the cable.

18. The method of claim 15, further comprising delivering fluid out of a distal port at the distal terminal end of the balloon.

19. The method of claim 15, further comprising actively or passively closing a configuration at the end of the first passageway distal to the antenna.

20. The method of claim 15, wherein the first passageway is at least partially surrounded by a liner, and wherein the first passageway is inside of a lumen in an inner conductor, wherein the lumen is defined by an inner conductor inner wall, and wherein the liner is not secured to the inner conductor inner wall around the entire radius of the inner conductor inner wall.

21. The method of claim 20, wherein the end of the first passageway distal to the emitter has fluid ports and/or pores proximal to a controllably closable configuration, and wherein the method further comprises closing the first passageway distal to the fluid ports or pores With the controllably closable configuration.

22. The method of claim 21, wherein the controllably closable configuration comprises a valve.

23. The method of claim 21, wherein the controllably closable configuration comprises an inflatable occluding balloon.

24. The method of claim 15, further comprising a second passageway inside the coaxial cable.

25. The method of claim 15, further comprising delivering, power to the emitter via a power supply, delivering the fluid is a fluid supply, delivering a guidewire through the emitter via a second passageway in the cable, attaching a connector to the proximal end of the cable, connecting the power supply and fluid supply to the connector, and inserting the guidewire through the connector.

26. The method of claim 15, wherein the cable has an inner conductor, an outer conductor, and one or more dielectrics between the inner conductor and the outer conductor, and wherein the first passageway extends through the one or more dielectrics.
